(19) United States
(12) Patent Application Publication
(10) Pub. No.: US 2013/0040914 A1
(43) Pub. Date: Feb. 14, 2013

(54) PRODRUGS OF AN HIV REVERSE TRANSCRIPTASE INHIBITOR

(71) Applicants: Samson M. Jolly, Holden, MA (US); Neville Anthony, Boston, MA (US); Robert Gomez, Boston, MA (US); David C. Dubost, Boston, MA (US); Rick G. Woodward, Boston, MA (US)

(72) Inventors: Samson M. Jolly, Holden, MA (US); Neville Anthony, Boston, MA (US); Robert Gomez, Boston, MA (US); David C. Dubost, Boston, MA (US); Rick G. Woodward, Boston, MA (US)

(21) Appl. No.: 13/639,954

(22) PCT Filed: Apr. 4, 2011
(86) PCT No.: PCT/US11/31070

§ 371 Date: Oct. 8, 2012

Related U.S. Application Data
(60) Provisional application No. 61/322,067, filed on Apr. 8, 2010.

(51) Int. Cl. A61K 31/675; A61K 31/444; C07D 471/04; C07F 9/6561; A61P 31/18

(52) U.S. Cl. 514/81; 546/23; 546/120; 514/303

(57) ABSTRACT

Compounds of Formula I are described: (I), wherein R^{P} and R^{Q} are defined herein. The compounds transcriptase, the prophylaxis and treatment of infection by HIV, and the prophylaxis, delay in the onset or progression, and treatment of AIDS. The compounds can be employed as ingredients in pharmaceutical compositions, optionally in combination with other antivirals, immunomodulators, antibiotics or vaccines.

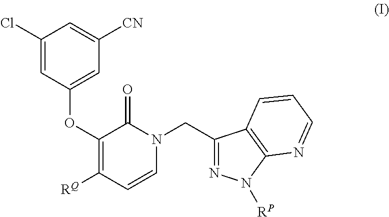

### FIELD OF THE INVENTION

The present invention is directed to certain derivatives of 3-(3,5-disubstituted phenoxy)-1-(1H-pyrazolo[3,4-b]pyridin-3-ylmethyl)-4-(substituted)pyridin-2(1H)-ones. The derivatives are useful, for example, in the inhibition of HIV reverse transcriptase, the prophylaxis of HIV infection and HIV replication, the treatment of HIV infection and HIV replication, the prophylaxis of AIDS, the treatment of AIDS, and the delay in the onset and/or progression of AIDS.

### BACKGROUND OF THE INVENTION

WO 2009/067166 A2 discloses certain aryloxy-, cycloalkyloxy-, and heterocyclyloxy-pyridines and pyrimidines and related compounds and their use for the inhibition of HIV reverse transcriptase. Among the compounds disclosed are certain 3-(3,5-disubstituted phenoxy)-1-(1H-pyrazolo[3,4-b]pyridin-3-ylmethyl)-4-(substituted)pyridin-2(1H)-ones. Representative of these compounds are 3-chloro-5-{[2-oxo-1-(1H-pyrazolo[3,4-b]pyridin-3-ylmethyl)-4-(trifluoromethyl)-1,2-dihydropyridin-3-yl]oxy}benzonitrile (Example 13 in WO '166; hereinafter “Compound A”), and 3-chloro-5-{[4-chloro-2-oxo-1-(1H-pyrazolo[3,4-b]pyridin-3-ylmethyl)-1,2-dihydropyridin-3-yl]oxy}benzonitrile (Example 21 in WO '166; hereinafter “Compound B”). Compounds A and B have the following structures:

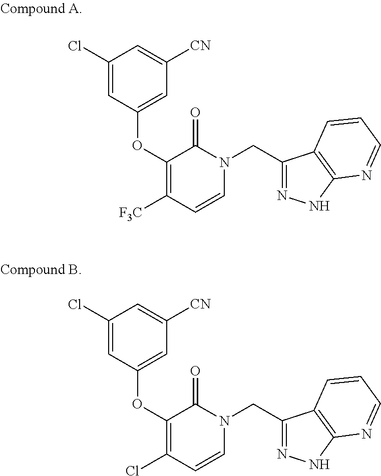 [SMILES: N#Cc1cc(Cl)cc(Oc2c(C(F)(F)F)ccn(Cc3n[nH]c4ncccc34)c2=O)c1 ; N#Cc1cc(Cl)cc(Oc2c(Cl)ccn(Cc3n[nH]c4ncccc34)c2=O)c1]

Compounds A and B are potent non-nucleoside reverse transcriptase inhibitors, but both have relatively low aqueous solubility, and Compound A has exhibited relatively low oral bioavailability in pharmacokinetic studies in animals.

WO 2009/067166 A2 discloses certain prodrugs of its compounds and states that the prodrugs can exhibit enhanced solubility, absorption and/or lipophilicity compared to the compounds per se.

### SUMMARY OF THE INVENTION

The present invention is directed to certain derivatives of 3-(3,5-disubstituted phenoxy)-1-(1H-pyrazolo[3,4-b]pyridin-3-ylmethyl)-4-(substituted)pyridin-2(1H)-ones. These compounds (including hydrates and solvates thereof) are useful in the inhibition of reverse transciptase in retroviruses, for the treatment or prophylaxis of retroviral infections, and for the prophylaxis or treatment of diseases or conditions resulting from retroviral infection. The compounds are useful, for example, in the inhibition of HIV reverse transcriptase, the prophylaxis of infection by HIV, the treatment of infection by HIV and in the prophylaxis, treatment, and delay in the onset or progression of AIDS and/or ARC, either as compounds per se, or as pharmaceutical composition ingredients, whether or not in combination with other HIV antivirals, anti-infectives, immunomodulators, antibiotics or vaccines. More particularly, the present invention includes compounds of Formula I:

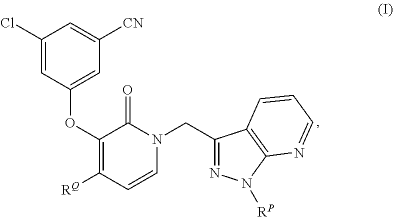

wherein:

### R^{P} is:

(1) a base salt of

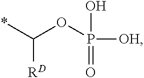

(2) an acid salt of C(O)N(R^{A})—C₁₋₆ alkylene-N(R^{A})R^{B};

(3) an acid salt of C(O)—C₁₋₆ alkylene-N(R^{A})R^{B}, or

(4) C(O)OR^{C};

R^{A} and R^{B} are each independently H or C₁₋₄ alkyl;
R^{C} is C₁₋₃ alkyl;

### R^{D} is H or CH₃; and

R^{Q} is CF₃ or halogen.

The term “base salt” as used herein with respect to part (1) of the definition of R^{P} refers to a salt which is represented by the loss of at least one proton from the group balanced by one or more positive counterions (e.g., an alkali metal cation). A base salt of (1) in the definition of R^{P} can be represented as:

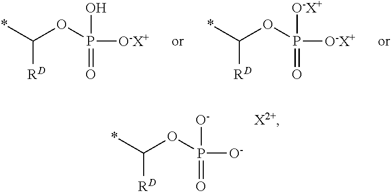

wherein X⁺ and X²⁺ are positive counterions. The base salt can be formed by treating the free form of the compound of Formula I with a suitable inorganic or organic base. Suitable inorganic bases include ammonium hydroxide, alkali metal hydroxides (e.g., NaOH or KOH), alkaline earth metal hydroxides and the like. Suitable organic bases include alkali metal alkylcarboxylates (e.g., potassium acetate or sodium acetate), alkyl ammonium hydroxides and the like.

The term “acid salt” as used herein with respect to parts (2) and (3) of the definition of R^{P} refers to a salt which is represented by the addition of a proton to the group balanced by a suitable negative counterion. An acid salt of (2) in the definition of R^{P} can be represented as:

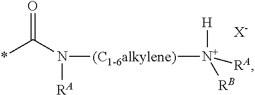

wherein X⁻ is the counterion. An acid salt of (3) can be represented as:

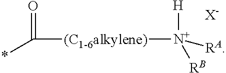

The acid salts are acid addition salts that can be formed by treating the free form of the compound of Formula I with an inorganic or organic acid. Suitable inorganic acids include hydrochloric acid, hydrobromic acid, hydrogen iodide, sulfuric acid, nitric acid, phosphoric acid, and the like. Suitable organic acids include organic sulfonic acids (e.g., alkylsulfonic acids such as methanesulfonic acid and arylsuflonic acids such as p-toluenesulfonic acid and benzenesulfonic acid), organic carboxylic acids (e.g., acetic acid, tartaric acid, maleic acid, critic acid, benzoic acid, salicylic acid and ascorbic acid), and the like.

The compounds of Formula I are believed to act as prodrugs which are converted in vivo into their pharmaceutically active counterparts of Formula I′, wherein Formula I′ is identical to Formula I, except that R^{P} is replaced with H. As noted in the Background of the Invention, compounds of Formula I′ are HIV reverse transcriptase inhibitors. Compounds of Formula I can exhibit significantly higher aqueous solubilities at physiological pH than their Formula I′ counterparts. Derivatives of Compound A embraced by Formula I are believed to be representative of compounds of Formula I. Several such derivatives have exhibited significantly higher oral bioavailabilities of Compound A in animal tests in comparison to values obtained in similar tests using Compound A directly. As noted in the Background of the Invention, WO 2009/067166 A2 discloses prodrugs, but it does not disclose the compounds of Formula I. It is further noted that N-phosphate prodrugs of the type described in WO '166 can be unstable. For example, attempts to isolate the N-phosphate of Compound A and salts thereof have been unsuccessful, whereas N-alkyl phosphate salts embraced by Formula I above have been isolated and are chemically stable.

The present invention also includes pharmaceutical compositions containing a compound of Formula I. The present invention further includes methods involving compounds of Formula I for the treatment of AIDS, the delay in the onset or progression of AIDS, the prophylaxis of AIDS, the prophylaxis of infection by HIV, and the treatment of infection by HIV.

Other embodiments, aspects and features of the present invention are either further described in or will be apparent from the ensuing description, examples and appended claims.

### BRIEF DESCRIPTION OF THE DRAWINGS

FIG. 1 is a plot of the thermogravimetric analysis for the product of Example 1.

FIG. 2 is the X-ray powder diffraction pattern for the product of Example 1.

FIG. 3 is the DSC curve for the product of Example 1.

FIG. 4 is a plot of the thermogravimetric analysis for the product of Example 9A.

FIG. 5 is the DSC curve for the product of Example 9A.

FIG. 6 is the X-ray powder diffraction pattern for the product of Example 9A.

### DETAILED DESCRIPTION OF THE INVENTION

The compounds of Formula I above are believed to be prodrugs which can be metabolized in vivo to compounds of Formula I′ (defined above) which are inhibitors of HIV reverse transcriptase. As further described in WO 2009/067166, the compounds of Formula I′inhibit the polymerase function of HIV-1 reverse transcriptase, and more particularly inhibit the RNA-dependent DNA polymerase activity of HIV-1 reverse transcriptase. The compounds of Formula I′ also exhibit activity against drug resistant forms of HIV (e.g., mutant strains of HIV-1 in which reverse transcriptase has a mutation at lysine 103→asparagine (K103N) and/or tyrosine 181→cysteine (Y181C)), and thus can exhibit decreased cross-resistance against currently approved antiviral therapies.

A first embodiment of the present invention (alternatively referred to herein as “Embodiment E1”) is a compound of Formula I, wherein R^{Q} is CF₃ or Cl; and all other variables are as originally defined above (i.e., as defined in the Summary of the Invention).

A second embodiment of the present invention (Embodiment E2) is a compound of Formula I (alternatively referred to herein as “Compound I”), wherein R^{Q} is CF₃; and all other variables are as originally defined above.

A third embodiment of the present invention (Embodiment E3) is a compound of Formula I, wherein R^{Q} is Cl; and all other variables are as originally defined above.

A fourth embodiment of the present invention (Embodiment E4) is a compound of Formula I, wherein R^{A} and R^{B} are each independently H or C₁₋₃ alkyl; and all other variables are as originally defined above or as defined in any of Embodiments E1 to E3.

A fifth embodiment of the present invention (Embodiment E5) is a compound of Formula I, wherein R^{A} and R^{B} are each independently H or CH₃; and all other variables are as originally defined or as defined in any of Embodiments E to E3.

A sixth embodiment of the present invention (Embodiment E6) is a compound of Formula I, wherein R^{C} is methyl or ethyl; and all other variables are as originally defined or as defined in any of the preceding embodiments. In a first aspect of Embodiment E6, R^{C} is CH₃. In a second aspect of this embodiment, R^{C} is CH₂CH₃.

A seventh embodiment of the present invention (Embodiment E7) is a compound of Formula I, wherein RD is H; and all other variables are as originally defined or as defined in any of the preceding embodiments.

An eighth embodiment of the present invention (Embodiment E8) is a compound of Formula I, wherein R^{D} is CH₃; and all other variables are as originally defined or as defined in any of the preceding embodiments.

A ninth embodiment of the present invention (Embodiment E9) is a compound of Formula I, wherein R^{P} is:
(1) an alkali metal salt, an alkaline earth metal salt, an ammonium salt, or a tetra (C₁₋₄ alkyl) ammonium salt of

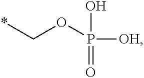 [SMILES: *COP(=O)(O)O]

(2) an alkali metal salt, an alkaline earth metal salt, an ammonium salt, or a tetra (C₁₋₄ alkyl) ammonium salt of

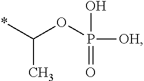 [SMILES: *C(C)OP(=O)(O)O]

(3) an acid salt of

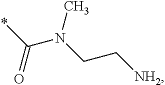 [SMILES: *C(=O)N(C)CCN]

(4) an acid salt of

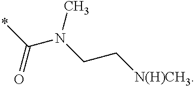

(5) C(O)CH₂N(CH₃)₂,
(6) C(O)C(CH₃)₂CH₂N(CH₃)₂,
(7) C(O)OCH₃, or
(8) C(O)OCH₂CH₃;
   and all other variables are as originally defined above or as defined in any one of Embodiments E1 to E3.

A tenth embodiment of the present invention (Embodiment E10) is a compound of Formula I, wherein R^{P} is an alkali metal salt, an alkaline earth metal salt, an ammonium salt, or a tetra (C₁₋₄ alkyl) ammonium salt of

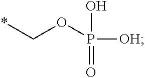 [SMILES: *COP(=O)(O)O]

and all other variables are as originally defined above or as defined in any one of Embodiments E1 to E3. In a first aspect of Embodiment E10, R^{P} is an ammonium salt or an alkali metal salt of *—CH₂OP(O)(OH)₂. In a second aspect of this embodiment, R^{P} is an ammonium salt of *—CH₂OP(O)(OH)₂. In a third aspect of this embodiment, R^{P} is an alkali metal salt of *—CH₂OP(O)(OH)₂. In a fourth aspect of this embodiment, R^{P} is a sodium salt of *—CH₂OP(O)(OH)₂. In a fifth aspect of this embodiment, R^{P} is a potassium salt of *—CH₂OP(O)(OH)₂. In a feature of the fifth aspect, the potassium salt of *—CH₂OP(O)(OH)₂ is a monopotassium salt or a dipotassium salt. In another feature of the fifth aspect, the potassium salt of *—CH₂OP(O)(OH)₂ is a monopotassium salt. In yet another feature of the fifth aspect, the potassium salt of *—CH₂OP(O)(OH)₂ is a monopotassium solvate salt. In yet another feature of the fifth aspect, the potassium salt of *—CH₂OP(O)(OH)₂ is a crystalline monopotassium monohydrate salt. In a sub-feature of this feature, R^{Q} is CF₃. In still another feature of the fifth aspect, the potassium salt of *—CH₂OP(O)(OH)₂ is a dipotassium salt.

An eleventh embodiment of the present invention (Embodiment E11) is a compound of Formula I, wherein R^{P} is an alkali metal salt an alkaline earth metal salt, an ammonium salt, or a tetra (C₁₋₄ alkyl) ammonium salt of

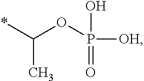 [SMILES: *C(C)OP(=O)(O)O]

and all other variables are as originally defined above or as defined in any one of Embodiments E1 to E3. In a first aspect of Embodiment E11, R^{P} is an ammonium salt or an alkali metal salt of *—CH(CH₃)OP(O)(OH)₂. In a second aspect of this embodiment, R^{P} is an ammonium salt of *—CH(CH₃)OP(O)(OH)₂. In a third aspect of this embodiment, R^{P} is an alkali metal salt of *—CH(CH₃)OP(O)(OH)₂. In a fourth aspect of this embodiment, R^{P} is a sodium salt of *—CH(CH₃)OP(O)(OH)₂. In a fifth aspect of this embodiment, R^{P} is a potassium salt of *—CH(CH₃)OP(O)(OH)₂. In a feature of the fifth aspect, the potassium salt of *—CH₂OP(O)(OH)₂ is a monopotassium salt or a dipotassium salt. In another feature of the fifth aspect, the potassium salt of *—CH(CH₃)OP(O)(OH)₂ is a monopotassium salt. In still another feature of the fifth aspect, the potassium salt of *—CH(CH₃)OP(O)(OH)₂ is a dipotassium salt.

A twelfth embodiment of the present invention (Embodiment E12) is a compound of Formula I, wherein R^{P} is an acid salt of

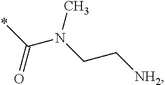 [SMILES: *C(=O)N(C)CCN]

and all other variables are as originally defined above or as defined in any one of Embodiments E1 to E3. In a first aspect of Embodiment E12, the acid salt is a hydrochloride salt, a besylate salt, a mesylate salt, a sulfonate salt, or a tosylate salt. In a second aspect of this embodiment, the acid salt is a hydrochloride salt. In a third aspect of this embodiment, the acid salt is a besylate salt. In a feature of this feature, R^{Q} is CF₃ and the besylate salt is a crystalline besylate monohydrate salt. In a third aspect of this embodiment, the acid salt is a mesylate salt. In a fourth aspect of this embodiment, the acid salt is a sulfonate salt. In a fifth aspect of this embodiment, the acid salt is a tosylate salt.

A thirteenth embodiment of the present invention (Embodiment E13) is a compound of Formula I, wherein R^{P} is an acid salt of

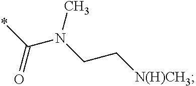

and all other variables are as originally defined above or as defined in any one of Embodiments E1 to E3. In a first aspect of Embodiment E13, the acid salt is a hydrochloride salt, a besylate salt, a mesylate salt, a sulfonate salt, or a tosylate salt. In a second aspect of this embodiment, the acid salt is a hydrochloride salt. In a third aspect of this embodiment, the acid salt is a besylate salt. In a third aspect of this embodiment, the acid salt is a mesylate salt. In a fourth aspect of this embodiment, the acid salt is a sulfonate salt. In a fifth aspect of this embodiment, the acid salt is a tosylate salt.

A fourteenth embodiment of the present invention (Embodiment E14) is a compound of Formula I, wherein R^{P} is C(O)CH₂N(CH₃)₂; and all other variables are as originally defined above or as defined in any one of Embodiments E1 to E3.

A fifteenth embodiment of the present invention (Embodiment E15) is a compound of Formula I, wherein R^{P} is C(O)C(CH₃)₂CH₂N(CH₃)₂ or C(O)CH₂CH₂N(CH₃)₂; and all other variables are as originally defined above or as defined in any one of Embodiments E1 to E3. In a first aspect of Embodiment E15, R^{P} is C(O)C(CH₃)₂CH₂N(CH₃)₂. In a second aspect of this embodiment, R^{P} is C(O)CH₂CH₂N(CH₃)₂.

A sixteenth embodiment of the present invention (Embodiment E16) is a compound of Formula I, wherein R^{P} is C(O)OCH₃; and all other variables are as originally defined above or as defined in any one of Embodiments E1 to E3.

A seventeenth embodiment of the present invention (Embodiment E17) is a compound of Formula I, wherein R^{P} is C(O)OCH₂CH₃; and all other variables are as originally defined above or as defined in any one of Embodiments E to E3.

An eighteenth embodiment of the present invention (Embodiment E18) is a compound of Formula I, wherein the compound is selected from the group consisting of the title compounds set forth in Examples 1 to 9A. In a first aspect of Embodiment E18, the compound is the compound of Example 1. In a second aspect of this embodiment, the compound is the compound of Example 2. In a third aspect of this embodiment, the compound is the compound of Example 3. In a fourth aspect of this embodiment, the compound is the compound of Example 4. In a fifth aspect of this embodiment, the compound is the compound of Example 5. In a sixth aspect of this embodiment, the compound is the compound of Example 6. In a seventh aspect of this embodiment, the compound is the compound of Example 7. In an eighth aspect of this embodiment, the compound is the compound of Example 8. In a ninth aspect of this embodiment, the compound is the compound of Example 9. In a tenth aspect of this embodiment, the compound is the compound of Example 9A.

A nineteenth embodiment of the present invention (Embodiment E19) is a compound of Formula I as originally defined or as defined in any of the foregoing embodiments, aspects, features, or sub-features, wherein the compound is in a substantially pure form. As used herein “substantially pure” means suitably at least about 60 wt. %, typically at least about 70 wt. %, preferably at least about 80 wt. %, more preferably at least about 90 wt. % (e.g., from about 90 wt. % to about 99 wt. %), even more preferably at least about 95 wt. % (e.g., from about 95 wt. % to about 99 wt. %, or from about 98 wt. % to 100 wt. %), and most preferably at least about 99 wt. % (e.g., 100 wt. %) of a product containing a compound of Formula I (e.g., the product isolated from a reaction mixture affording the compound) consists of the compound. The level of purity of the compounds can be determined using a standard method of analysis such as thin layer chromatography, gel electrophoresis, high performance liquid chromatography, and/or mass spectrometry. If more than one method of analysis is employed and the methods provide experimentally significant differences in the level of purity determined, then the method providing the highest purity level governs. A compound of 100% purity is one which is free of detectable impurities as determined by a standard method of analysis. With respect to a compound of the invention which has one or more asymmetric centers and can occur as mixtures of stereoisomers, a substantially pure compound can be either a substantially pure mixture of the stereoisomers or a substantially pure individual diastereomer or enantiomer.

Other embodiments of the present invention include the following:

(a) A pharmaceutical composition comprising a compound of Formula I (e.g., an effective amount of Compound I) as defined above and a pharmaceutically acceptable carrier.

(b) A pharmaceutical composition which comprises the product prepared by combining (e.g., mixing) a compound of Formula I as defined above and a pharmaceutically acceptable carrier.

(c) The pharmaceutical composition of (a) or (b), further comprising an effective amount of an anti-HIV agent selected from the group consisting of HIV antiviral agents, immunomodulators, and anti-infective agents.

(d) The pharmaceutical composition of (c), wherein the anti-HIV agent is an antiviral selected from the group consisting of HIV protease inhibitors, nucleoside HIV reverse transcriptase inhibitors, non-nucleoside HIV reverse transcriptase inhibitors, HIV integrase inhibitors, HIV fusion inhibitors, and HIV entry inhibitors.

(e) A combination which is (i) a compound of Formula I as defined above and

(ii) an anti-HIV agent selected from the group consisting of HIV antiviral agents, immunomodulators, and anti-infective agents; wherein Compound I and the anti-HIV agent are each employed in an amount that renders the combination effective for inhibition of HIV reverse transcriptase, for treatment or prophylaxis of infection by HIV, or for treatment, prophylaxis of, or delay in the onset or progression of AIDS.

(f) The combination of (e), wherein the anti-HIV agent is an antiviral selected from the group consisting of HIV protease inhibitors, nucleoside HIV reverse transcriptase inhibitors, non-nucleoside HIV reverse transcriptase inhibitors, HIV integrase inhibitors, HIV fusion inhibitors, and HIV entry inhibitors.

(g) A method for the inhibition of HIV reverse transcriptase in a subject in need thereof which comprises administering to the subject an effective amount of a compound of Formula I.

(h) A method for the prophylaxis or treatment of infection by HIV (e.g., HIV-1) in a subject in need thereof which comprises administering to the subject an effective amount of a compound of Formula I.

(i) The method of (h), wherein the compound of Formula I is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, and HIV entry inhibitors.

(j) A method for the prophylaxis, treatment or delay in the onset or progression of AIDS in a subject in need thereof which comprises administering to the subject an effective amount of a compound of Formula I.

(k) The method of (j), wherein the compound is administered in combination with an effective amount of at least one other HIV antiviral selected from the group consisting of HIV protease inhibitors, HIV integrase inhibitors, non-nucleoside HIV reverse transcriptase inhibitors, nucleoside HIV reverse transcriptase inhibitors, HIV fusion inhibitors, and HIV entry inhibitors.

(l) A method for the inhibition of HIV reverse transcriptase in a subject in need thereof which comprises administering to the subject the pharmaceutical composition of (a), (b), (c) or (d) or the combination of (e) or (f).

(m) A method for the prophylaxis or treatment of infection by HIV (e.g., HIV-1) in a subject in need thereof which comprises administering to the subject the pharmaceutical composition of (a), (b), (c) or (d) or the combination of (e) or (f).

(n) A method for the prophylaxis, treatment, or delay in the onset or progression of AIDS in a subject in need thereof which comprises administering to the subject the pharmaceutical composition of (a), (b), (c) or (d) or the combination of (e) or (f).

The present invention also includes a compound of Formula I (i) for use in, (ii) for use as a medicament for, or (iii) for use in the preparation of a medicament for: (a) therapy (e.g., of the human body), (b) medicine, (c) inhibition of HIV reverse transcriptase, (d) treatment or prophylaxis of infection by HIV, or (e) treatment, prophylaxis of, or delay in the onset or progression of AIDS. In these uses, the compounds of the present invention can optionally be employed in combination with one or more anti-HIV agents selected from HIV antiviral agents, anti-infective agents, and immunomodulators.

Additional embodiments of the invention include the pharmaceutical compositions, combinations and methods set forth in (a)-(n) above and the uses (i)(a)-(e) through (iii)(a)-(e) set forth in the preceding paragraph, wherein the compound of the present invention employed therein is a compound of one of the embodiments, aspects, features, or sub-features described above.

Additional embodiments of the present invention include each of the pharmaceutical compositions, combinations, methods and uses set forth in the preceding paragraphs, wherein the compound of the present invention employed therein is substantially pure. With respect to a pharmaceutical composition comprising a compound of Formula I and a pharmaceutically acceptable carrier and optionally one or more excipients, it is understood that the term “substantially pure” is in reference to a compound of Formula I per se.

Still additional embodiments of the present invention include the pharmaceutical compositions, combinations and methods set forth in (a)-(n) above and the uses (i)(a)-(e) through (iii)(a)-(e) set forth above, wherein the HIV of interest is HIV-1. Thus, for example, in the pharmaceutical composition (d), the compound of Formula I is employed in an amount effective against HIV-1 and the anti-HIV agent is an HIV-1 antiviral selected from the group consisting of HIV-1 protease inhibitors, HIV-1 reverse transcriptase inhibitors, HIV-1 integrase inhibitors, HIV-1 fusion inhibitors and HIV-1 entry inhibitors.

As used herein, the term “alkyl” refers to a monovalent straight or branched chain, saturated aliphatic hydrocarbon radical having a number of carbon atoms in the specified range. Thus, for example, “C₁₋₄ alkyl” refers to n-, iso-, sec- and t-butyl, n- and isopropyl, ethyl and methyl.

The term “alkylene” refers to any divalent linear or branched chain aliphatic hydrocarbon radical having a number of carbon atoms in the specified range. Thus, for example, “—C₁₋₆ alkylene-” refers to any of the C₁ to C₆ linear or branched alkylenes, and “—C₁₋₄ alkylene-” refers to any of the C₁ to C₄ linear or branched alkylenes. A class of alkylenes of interest with respect to the invention is —(CH₂)₁₋₆—. Another class of alkylenes of interest with respect to the invention is —(CH₂)₁₋₄—. Another class of interest is an alkylene selected from the group consisting of —CH₂—, —CH(CH₃)—, and —C(CH₃)₂—.

The term “halogen” (or “halo”) refers to fluorine, chlorine, bromine and iodine (alternatively referred to as fluoro, chloro, bromo, and iodo).

The term “C(O)” refers to carbonyl.

An asterisk (“*”) as the end of an open bond in a chemical group denotes the point of attachment of the group to the rest of the compound.

Unless expressly stated to the contrary, all ranges cited herein are inclusive. For example, a dosage in a range of 1 to 500 milligrams means the dosage can be 1 mg or 500 mg or any amount therebetween.

When any variable (e.g., R^{A} or R^{B}) occurs more than one time in any constituent or in Formula I or in any other formula depicting and describing compounds of the present invention, its definition on each occurrence is independent of its definition at every other occurrence. Also, combinations of substituents and/or variables are permissible only if such combinations result in stable compounds.

The present invention includes solvates of the compounds of Formula I. The term “solvate” refers to a form of a compound in which the compound is associated with a pharmaceutically acceptable solvent. Solvates can be formed via a solvolysis reaction. Representative solvents include water, alkyl alcohols (e.g., methanol or ethanol), carboxylic acids (e.g., acetic acid), and the like. When water is the solvent, the compound is alternatively referred to as a hydrate. The solvates include both stoichiometric and non-stoichiometric solvates.

As would be recognized by one of ordinary skill in the art, certain of the compounds described herein can exist as tautomers. For example, compounds of Formula I′ can exist as tautomers:

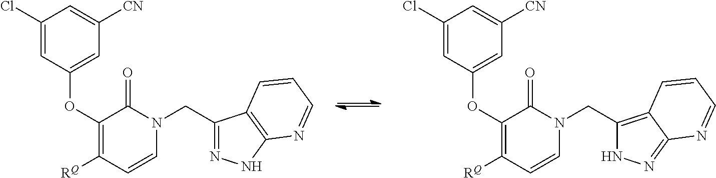

It is understood that a reference to a compound capable of tautomerism includes within its scope a reference to each individual tautomer and combinations thereof.

A “stable” compound is a compound which can be prepared and isolated and whose structure and properties remain or can be caused to remain essentially unchanged for a period of time sufficient to allow use of the compound for the purposes described herein (e.g., therapeutic or prophylactic administration to a subject). The compounds of the present invention are limited to stable compounds embraced by Formula I.

As a result of the selection of substituents and substituent patterns, certain compounds of the present invention can have asymmetric centers and can occur as mixtures of stereoisomers, or as individual diastereomers, or enantiomers. All isomeric forms of these compounds, whether individually or in mixtures, are within the scope of the present invention.

The atoms in a compound of Formula I may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of generic Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within generic Formula I can be prepared without undue experimentation by conventional techniques well known to those skilled in the art or by processes analogous to those described in the Schemes and Examples herein using appropriate isotopically-enriched reagents and/or intermediates.

The methods of the present invention involve the use of compounds of the present invention in the inhibition of HIV reverse transciptase (e.g., wild type HIV-1 and/or mutant strains thereof), the prophylaxis or treatment of infection by HIV and the prophylaxis, treatment or delay in the onset or progression of consequent pathological conditions such as AIDS. Preventing AIDS, treating AIDS, delaying the onset or progression of AIDS, or treating or preventing infection by HIV is defined as including, but not limited to, treatment of a wide range of states of HIV infection: AIDS, ARC, both symptomatic and asymptomatic, and actual or potential exposure to HIV. For example, the present invention can be employed to treat infection by HIV after suspected past exposure to HIV by such means as blood transfusion, exchange of body fluids, bites, accidental needle stick, or exposure to patient blood during surgery. As another example, the present invention can also be employed to prevent transmission of HIV from a pregnant female infected with HIV to her unborn child or from an HIV-infected female who is nursing (i.e., breast feeding) a child to the child via administration of an effective amount of Compound I.

Certain of the compounds of the invention are acid salts, and certain others are base salts. It is understood that these salts are pharmaceutically acceptable salts. The term “pharmaceutically acceptable salt” refers to a salt which possesses the effectiveness of the parent compound and which is not biologically or otherwise undesirable (e.g., is neither toxic nor otherwise deleterious to the recipient thereof).

The term “administration” and variants thereof (e.g., “administering” a compound) in reference to a compound of Formula I mean providing the compound to the individual in need of treatment or prophylaxis. When a compound is provided in combination with one or more other active agents (e.g., antiviral agents useful for treating or prophylaxis of HIV infection or AIDS), “administration” and its variants are each understood to include provision of the compound and other agents at the same time or at different times. When the agents of a combination are administered at the same time, they can be administered together in a single composition or they can be administered separately.

As used herein, the term “composition” is intended to encompass a product comprising the specified ingredients, as well as any product which results, directly or indirectly, from combining the specified ingredients.

Ingredients suitable for inclusion in a pharmaceutical composition are pharmaceutically acceptable ingredients, which means the ingredients must be compatible with each other and not deleterious to the recipient thereof.

The term “subject” as used herein refers to an animal, preferably a mammal, most preferably a human, who has been the object of treatment, observation or experiment.

The term “effective amount” as used herein means that amount of Compound I or other pharmaceutical agent that elicits the biological or medicinal response in a tissue, system, animal or human that is being sought by a researcher, veterinarian, medical doctor or other clinician. In one embodiment, the effective amount is a “therapeutically effective amount” for the alleviation of the symptoms of the disease or condition being treated. In another embodiment, the effective amount is a “prophylactically effective amount” for prophylaxis of the symptoms of the disease or condition being prevented. The term also includes herein the amount of Compound I sufficient to inhibit HIV reverse transcriptase (wild type and/or mutant strains thereof) and thereby elicit the response being sought (i.e., an “inhibition effective amount”).

In the method of the present invention (i.e., inhibiting HIV reverse transcriptase, treating or prophylaxis of HIV infection or treating, prophylaxis of, or delaying the onset or progression of AIDS), the compounds of Formula I can be administered by any means that results contact of the active agent with the agent's site of action. They can be administered by any conventional means available for use in conjunction with pharmaceuticals, either as individual therapeutic agents or in a combination of therapeutic agents. They can be administered alone, but typically are administered with a pharmaceutical carrier selected on the basis of the chosen route of administration and standard pharmaceutical practice. The compounds of the invention can, for example, be administered orally, parenterally (including subcutaneous injections, intravenous, intramuscular, intrasternal injection or infusion techniques), by inhalation spray, or rectally, in the form of a unit dosage of a pharmaceutical composition containing an effective amount of the compound and conventional non-toxic pharmaceutically acceptable carriers, adjuvants and vehicles. Liquid preparations suitable for oral administration (e.g., suspensions, syrups, elixirs and the like) can be prepared according to techniques known in the art and can employ any of the usual media such as water, glycols, oils, alcohols and the like. Solid preparations suitable for oral administration (e.g., powders, pills, capsules and tablets) can be prepared according to techniques known in the art and can employ such solid excipients as starches, sugars, kaolin, lubricants, binders, disintegrating agents and the like. Parenteral compositions can be prepared according to techniques known in the art and typically employ sterile water as a carrier and optionally other ingredients, such as a solubility aid. Injectable solutions can be prepared according to methods known in the art wherein the carrier comprises a saline solution, a glucose solution or a solution containing a mixture of saline and glucose. Further description of methods suitable for use in preparing pharmaceutical compositions for use in the present invention and of ingredients suitable for use in said compositions is provided in Remington's Pharmaceutical Sciences, 18^{th} edition, edited by A. R. Gennaro, Mack Publishing Co., 1990 and in Remington—The Science and Practice of Pharmacy, 21st edition, Lippincott Williams & Wilkins, 2005.

The compounds of Formula I can be administered orally in a dosage range of 0.001 to 1000 mg/kg of mammal (e.g., human) body weight per day in a single dose or in divided doses. One preferred dosage range is 0.01 to 500 mg/kg body weight per day orally in a single dose or in divided doses. Another preferred dosage range is 0.1 to 100 mg/kg body weight per day orally in single or divided doses. For oral administration, the compositions can be provided in the form of tablets or capsules containing 1 to 500 milligrams of a compound of the invention, particularly 1, 5, 10, 15, 20, 25, 50, 75, 100, 150, 200, 250, 300, 400, and 500 milligrams for the symptomatic adjustment of the dosage to the patient to be treated. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Unless expressly stated to the contrary, references in the preceding paragraph or elsewhere herein to the administration of a quantity of a compound of the invention are references to the amount of the corresponding compound of Formula I′. For example, a reference to the administration of 1000 mg/kg of a compound of the invention means an amount of Compound I which provides 1000 mg/kg of Compound I′. Thus, a reference to the administration of 1000 mg/kg of the compound of Example 1 means the amount of that compound which provides 1000 mg/kg of Compound A; i.e., 1330 mg/kg.

As noted above, the present invention is also directed to use of a compound of Formula I with one or more anti-HIV agents. An “anti-HIV agent” is any agent which is directly or indirectly effective in the inhibition of HIV reverse transcriptase or another enzyme required for HIV replication or infection, the treatment or prophylaxis of HIV infection, and/or the treatment, prophylaxis or delay in the onset or progression of AIDS. It is understood that an anti-HIV agent is effective in treating, preventing, or delaying the onset or progression of HIV infection or AIDS and/or diseases or conditions arising therefrom or associated therewith. For example, the compounds of this invention may be effectively administered, whether at periods of pre-exposure and/or post-exposure, in combination with effective amounts of one or more anti-HIV agents selected from HIV antiviral agents, immunomodulators, antiinfectives, or vaccines useful for treating HIV infection or AIDS. Suitable HIV antivirals for use in combination with the compounds of the present invention include, for example, those listed in Table A as follows:

TABLE A

| Name | Type |
|---|---|
| abacavir, ABC, Ziagen ® | nRTI |
| abacavir + lamivudine, Epzicom ® | nRTI |
| abacavir + lamivudine + zidovudine, Trizivir ® | nRTI |
| amprenavir, Agenerase ® | PI |
| atazanavir, Reyataz ® | PI |
| AZT, zidovudine, azidothymidine, Retrovir ® | nRTI |
| darunavir, Prezista ® | PI |
| ddC, zalcitabine, dideoxycytidine, Hivid ® | nRTI |
| ddI, didanosine, dideoxyinosine, Videx ® | nRTI |
| ddI (enteric coated), Videx EC ® | nRTI |
| delavirdine, DLV, Rescriptor ® | nnRTI |
| efavirenz, EFV, Sustiva ®, Stocrin ® | nnRTI |
| efavirenz + emtricitabine + tenofovir DF, | nRTI + nRTI |
| Atripla ® | |
| emtricitabine, FTC, Emtriva ® | nRTI |
| emtricitabine + tenofovir DF, Truvada ® | nRTI |
| emvirine, Coactinon ® | nnRTI |
| enfuvirtide, Fuzeon ® | FI |
| enteric coated didanosine, Videx EC ® | nRTI |
| etravirine, TMC-125, Intelence ® | nnRTI |
| fosamprenavir calcium, Lexiva ® | PI |
| indinavir, Crixivan ® | PI |
| lamivudine, 3TC, Epivir ® | nRTI |
| lamivudine + zidovudine, Combivir ® | nRTI |
| lopinavir | PI |
| lopinavir + ritonavir, Kaletra ® | PI |
| maraviroc, Selzentry ® | EI |
| nelfinavir, Viracept ® | PI |
| nevirapine, NVP, Viramune ® | nnRTI |
| raltegravir, MK-0518, Isentress ™ | InI |
| ritonavir, Norvir ® | PI |
| saquinavir, Invirase ®, Fortovase ® | PI |
| stavudine, d4T, didehydrodeoxythymidine, | nRTI |
| Zerit ® | |
| tenofovir DF (DF = disoproxil fumarate), | nRTI |
| TDF, Viread ® | |
| tipranavir, Aptivus ® | PI |
| vicriviroc | EI |

EI = entry inhibitor; FI = fusion inhibitor; InI = integrase inhibitor; PI = protease inhibitor; nRTI = nucleoside reverse transcriptase inhibitor; nnRTI = non-nucleoside reverse transcriptase inhibitor. Some of the drugs listed in the table are used in a salt form; e.g., abacavir sulfate, delavirdine mesylate, indinavir sulfate, atazanavir sulfate, nelfinavir mesylate, saquinavir mesylate.

It is understood that the scope of combinations of the compounds of this invention with anti-HIV agents is not limited to the HIV antivirals listed in Table A, but includes in principle any combination with any pharmaceutical composition useful for the treatment or prophylaxis of HIV infection or AIDS. The HIV antiviral agents and other agents will typically be employed in these combinations in their conventional dosage ranges and regimens as reported in the art, including, for example, the dosages described in editions of the Physicians' Desk Reference, such as the 63rd edition (2009) and earlier editions. The dosage ranges for a compound of the invention in these combinations can be the same as those set forth above.

While not wishing to be bound by any particular theory, it is believed that the compounds of the present invention act as prodrugs, wherein the compound is relatively stable at low pH (e.g., pH=1 to 3) but will convert by hydrolysis or cyclization to its free base at physiological pH (e.g., a pH of about 7), thereby releasing the active substance in vivo. For compounds of the invention in which R^{P} is an acid salt of C(O)N(R^{A})—CH₂CH₂—N(R^{A})R^{B}, it is believed the conversion can be depicted as follows:
1) When R^{A′}=H or alkyl and R^{B′}=H:

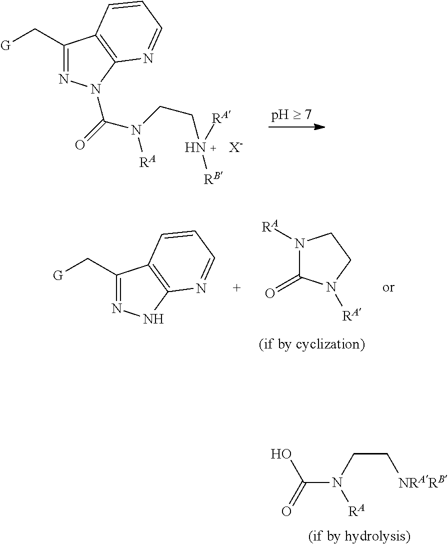

2) when R^{A′} and R^{B′}=alkyl:

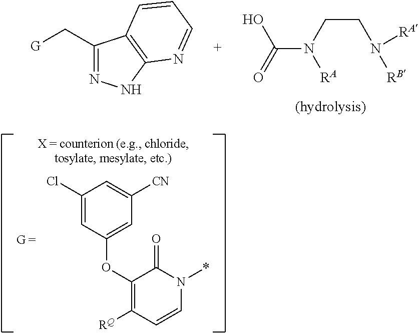

For compounds of the invention in which R^{P} is a alkyl phosphate base salt, it is believed that the phosphate group is cleaved primarily in the intestines by phosphatase enzymes in the lumen and secondarily at the brush border by phosphatases releasing the active substance in vivo. The conversion can be depicted as follows:

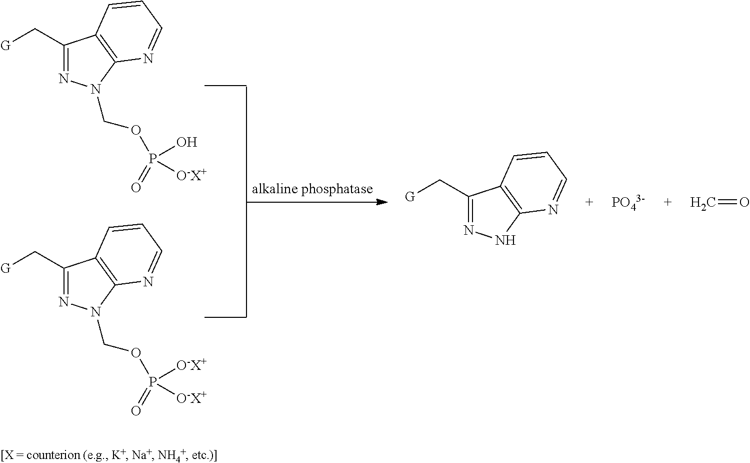

For compounds of the invention in which R^{P} is an aminoalkylcarbonyl group, it is believed that the group is cleaved by an amidase, a peptidase, and/or an esterase in the intestines, intracellularly after absorption and/or in the plasma, releasing the active substance in vivo. The conversion can be depicted as follows:

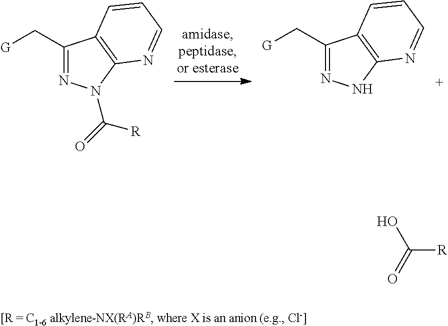

It is believed that compounds in which in which R^{P} is an alkoxycarbonyl group are cleaved by an esterase in the intestines, intracellularly after absorption and/or in the plasma, depicted as follows:

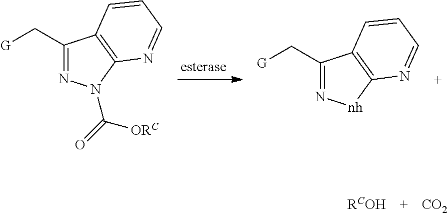

Abbreviations employed herein include the following:

AIDS=acquired immunodeficiency syndrome;
ARC=AIDS related complex;

### DMA=N,N-dimethylacetamide;

DMSO=dimethylsulfoxide;
Et=ethyl;
HIV=human immunodeficiency virus;
HPLC=high performance liquid chromatography;
LRMS=low resolution mass spectroscopy;
LC-MS=liquid chromatography-mass spectroscopy;
Me=methyl;

### NMP=N-methylpyrrolidinone

NMR=nuclear magnetic resonance;
TFA=trifluoroacetic acid;
TGA=thermogravimetric analysis;
THF=tetrahydrofuran;
TLC=thin layer chromatography.

The following examples serve only to illustrate the invention and its practice. The examples are not to be construed as limitations on the scope or spirit of the invention. In these examples, the term “room temperature” refers to a temperature in a range of from about 20° C. to about 25° C.

Compound A was prepared using the procedure described in Example 13 in WO 2009/067166, except for a minor modification in the method used to synthesize the reactant employed in Step 1. The reactant in Step 1 was 3-chloro-5-{[2-oxo-4-(trifluoromethyl)-1,2-dihydropyridin-3-yl]oxy}benzonitrile, which is referred to as 3C in WO '166. Compound 3C was prepared following the procedure set forth in Preparative Example 3 in WO 2009/067166, except that Step 3 therein was replaced with the following modified step:

A suspension of 3-(3-bromo-5-chlorophenoxy)-4-(trifluoromethyl)pyridin-2(1H)-one (10 g, 27.1 mmol), zinc (0.089 g, 1.357 mmol), palladium acetate (0.305 g, 1.357 mmol) and 1,1′-bis(diphenylphosphino)ferrocene (0.827 g, 1.492 mmol) in DMA (560 mL) was degassed, placed under N₂, and then charged with zinc cyanide (1.593 g, 13.57 mmol). This mixture was placed in an oil bath maintained at 90° C. for 16 hours, after which the reaction mixture was allowed to cool to room temperature. Water (200 mL) was added and the mixture was filtered, washing the solids with water. The solids were allowed to air dry and were crystallized from acetonitrile (320 mL) to give Compound 3C.

The preparative examples provide trifluoroacetate salts which can be converted to compounds of the invention by replacing trifluoroacetate with a suitable anion such as chloride. The TFA salts can be treated with a suitable acid in an amount and for a time sufficient to convert the TFA salt to the desired acid salt. Examples 6 to 9 exemplify the conversion of the TFA salt of Preparative Example 3 to salts of the present invention.

### Preparative Example 1

### 3-(3-{[3-(3-Chloro-5-cyanophenoxy)-2-oxo-4-(trifluoromethyl)pyridin-1(2H)-yl]methyl}-1H-pyrazolo[3,4-b]pyridin-1-yl)-N,N,2,2-tetramethyl-3-oxopropan-1-aminium trifluoroacetate

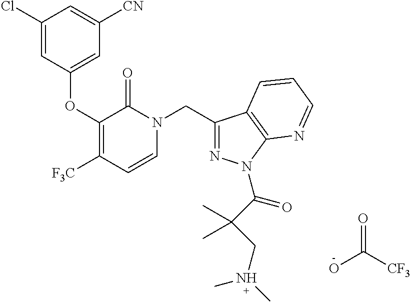 [SMILES: CN(C)CC(C)(C)C(=O)n1nc(Cn2ccc(C(F)(F)F)c(Oc3cc(Cl)cc(C#N)c3)c2=O)c2cccnc21 ; O=C(O)C(F)(F)F]

### Step 1: 3-Chloro-N,N,2,2-tetramethyl-3-oxopropan-1-aminium chloride

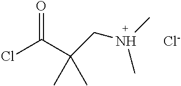

2-carboxy-N,N-dimethylpropan-2-aminium chloride (113 mg, 0.674 mmol) was dissolved in thionyl chloride (3 mL). The solution was stirred for 1 hour, after which solvent was evaporated therefrom in vacuo. The resulting oil was suspended in toluene, after which solvent was evaporated therefrom in vacuo to provide the title compound as a white solid. ¹H NMR (CDCl₃) δ 12.65 (br s, 1H), 3.40 (s, 2H), 2.85 (s, 6H), 1.64 (s, 6H).

### Step 2: 3-(3-{[3-(3-Chloro-5-cyanophenoxy)-2-oxo-4-(trifluoromethyl)pyridin-1(2H)-yl]methyl}-1H-pyrazolo[3,4-b]pyridin-1-yl)-N,N,2,2-tetramethyl-3-oxopropan-1-aminium trifluoroacetate

To 3-chloro-5-{[2-oxo-1-(1H-pyrazolo[3,4-b]pyridin-3-ylmethyl)-4-(trifluoromethyl)-1,2-dihydropyridin-3-yl]oxy}benzonitrile (50.0 mg, 0.112 mmol) in pyridine (2 mL) was added to 3-chloro-N,N,2,2-tetramethyl-3-oxopropan-1-aminium chloride (22.4 mg, 0.112 mmol) in pyridine (1 mL) at room temperature. N,N-dimethylpyridin-4-amine (13.7 mg, 0.112 mmol) was then added and the mixture was heated to 80° C. After 2 hours at 80° C., the reaction mixture was allowed to cool to room temperature, after which solvent was removed from the mixture by evaporation in vacuo. The resulting residue was purified by preparative HPLC eluting with 5-95% MeCN/H₂O+0.1% TFA. The desired product fraction was lyophilized to yield the title compound. ¹H NMR (DMSO-d6) δ 8.82 (d, J=3.8 Hz, 1H), 8.26 (d, J=6.7 Hz, 1H), 7.66 (d, J=7.3 Hz, 1H), 7.42 (dd, J=8.0 Hz, J=4.8 Hz, 1H), 7.38 (m, 1H), 7.16 (m, 1H), 7.02 (m, 1H), 6.54 (d, J=6.4 Hz, 1H), 5.55 (s, 2H), 3.62 (s, 2H), 2.96 (s, 6H), 1.78 (s, 6H). HRMS (M+1)=573.1635.

### Preparative Example 2

### 2-(3-{[3-(3-Chloro-5-cyanophenoxy)-2-oxo-4-(trifluoromethyl)pyridin-1(2H)-yl]methyl}-1H-pyrazolo[3,4-b]pyridin-1-yl)-N,N-dimethyl-2-oxoethanaminium trifluoroacetate

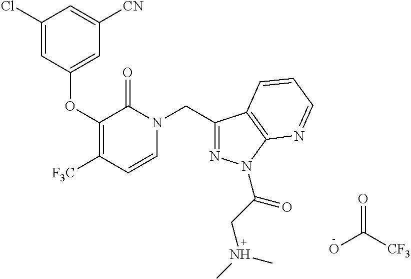 [SMILES: CN(C)CC(=O)n1nc(Cn2ccc(C(F)(F)F)c(Oc3cc(Cl)cc(C#N)c3)c2=O)c2cccnc21 ; O=C(O)C(F)(F)F]

N,N-Dimethylglycine (69.4 mg, 0.673 mmol), 1H-benzotriazol-1-ol (91 mg, 0.673 mmol) and N-[3-(dimethylamino)propyl]-N′-ethylcarbodiimide hydrochloride (129 mg, 0.673 mmol) were suspended in NMP (2 mL) with stirring. Once a solution was achieved, 3-chloro-5-{[2-oxo-1-(1H-pyrazolo[3,4-b]pyridin-3-ylmethyl)-4-(trifluoromethyl)-1,2-dihydropyridin-3-yl]oxy}benzonitrile (100 mg, 0.224 mmol) as a solution in NMP (2 mL) was added. N,N-dimethylpyridin-4-amine (82 mg, 0.673 mmol) was added. After 30 minutes TFA was added until a solution was achieved. This mixture was then purified by preparative HPLC eluting with 5-95% MeCN/H₂O+0.1% TFA and was lyophilized to afford the title compound. ¹H NMR (DMSO-d6) δ 8.78 (d, J=3.4 Hz, 1H), 8.13 (dd, J=8.0 Hz, J=1.5 Hz, 1H), 7.51 (d, J=7.0 Hz, 1H), 7.45 (dd, J=8.1 Hz, J-4.8 Hz, 1H), 7.38 (m, 1H), 7.16 (m, 1H), 6.97 (m, 1H), 6.53 (d, J=7.3 Hz, 1H), 5.52 (s, 2H), 4.99 (s, 2H), 3.21 (s, 6H). HRMS (M+1)=531.1154.

### Preparative Example 3

### 2-{[(3-{[3-(3-Chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethanaminium trifluoroacetate

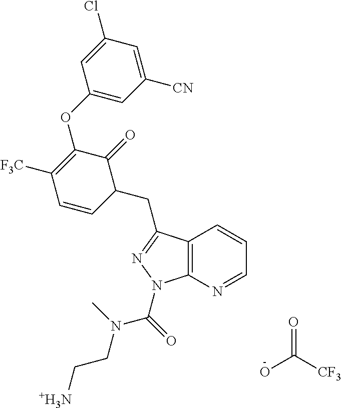 [SMILES: CN(CC[NH3+])C(=O)n1nc(CC2C=CC(C(F)(F)F)=C(Oc3cc(Cl)cc(C#N)c3)C2=O)c2cccnc21 ; O=C(O)C(F)(F)F]

### Step 1: tert-Butyl (2-{[(3-{[3-(3-chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethyl)carbamate

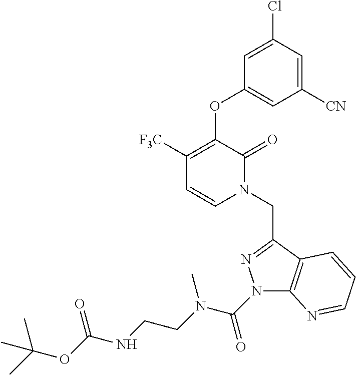 [SMILES: CN(CCNC(=O)OC(C)(C)C)C(=O)n1nc(Cn2ccc(C(F)(F)F)c(Oc3cc(Cl)cc(C#N)c3)c2=O)c2cccnc21]

3-Chloro-5-{[2-oxo-1-(1H-pyrazolo[3,4-b]pyridin-3-ylmethyl)-4-(trifluoromethyl)-1,2-dihydropyridin-3-yl]oxy}benzonitrile (6.0 g, 13.46 mmol) was stirred in DMA (140 mL) until dissolved and then cooled over ice bath for 10 minutes. This solution was treated with diisopropylethylamine (4.70 mL, 26.9 mmol) and then 4-nitrophenyl chloroformate (3.26 g, 16.15 mmol) portionwise over 5 minutes. This mixture was allowed to warm to room temperature and then stirred for 30 minutes. Formation of the intermediate carbamate was monitored by queching a small aliquot of the reaction mixture with dimethylamine and monitoring by LC-MS. The reaction mixture was then recooled over an ice bath and the cooled mixture was treated with a solution of 2-[(tert-butoxycarbonyl)amino]-N-methylethanaminium chloride (3.40 g, 16.15 mmol) and diisopropylethylamine (2.5 mL, 14.3 mmol) in DMA (35 mL). Upon completion of addition, the cooling bath was removed and the mixture stirred for 1 hour at room temperature. This mixture was partitioned between ethyl acetate (1000 mL and 500 mL) and water (500 mL). The combined extracts were further washed with water (3×500 mL), dried over MgSO₄, filtered and the solvent removed by evaporation in vacuo. This residue was pre-absorbed onto silica gel (35 g) using ethyl acetate and purified by silica gel (330 g) chromatography eluting with 0-100% ethyl acetate in hexanes. The desired fractions were combined and then solvent was removed by evaporation in vacuo. The resulting residue was further purified by re-crystallizing from ethyl acetate (75 mL) to give the title compound as a white solid. ¹H NMR (CDCl₃) δ=8.68 (d, 1H), 8.26 (d, 1H, J=7 Hz), 7.72 (d, 1H), 7.39 (dd, 1H, J=1.5 Hz), 7.30 (dd, 1H, J=4.6 and 8 Hz), 7.16 (dd, 1-i, J=2 Hz), 7.02 (dd, 1H, J=1.3 and 2.3 Hz), 6.46 (d, 1H, J=7 Hz), 5.18 (bs t, 1H), 5.50 (s, 2H), 3.7 (br, 2H), 3.52 (br, 2H), 3.17 (s, 3H) and 1.44 (s, 9H) ppm. LRMS (M+1): 646.1

### Step 2: 2-{[(3-{[3-(3-Chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethanaminium trifluoroacetate

tert-Butyl (2-{[(3-{[3-(3-chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethyl)carbamate (3.46 g, 5.98 mmol) was dissolved in TFA (40 mL) and allowed to stand at room temperature for 5 minutes, after which solvent was removed by evaporation in vacuo, and the resulting residue was purified by reverse phase chromatography using a LUNA C18 column (10μ, 250×30 mm) eluting with 5-95% aqueous acetonitrile with 0.1% TFA at 500 cmg/run. The desired fractions were combined and lyophilized to give the title compound. LRMS (M+1): 546.1.

### Example C-1

### 3-({1-[(1-Acetyl-H-pyrazolo[3,4-b]pyridin-3-yl)methyl]-2-oxo-4-(trifluoromethyl)-1,2-dihydropyridin-3-yl}oxy)-5-chlorobenzonitrile

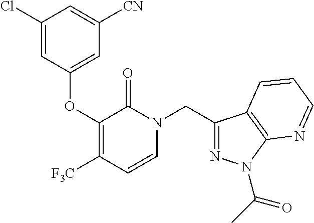 [SMILES: CC(=O)n1nc(Cn2ccc(C(F)(F)F)c(Oc3cc(Cl)cc(C#N)c3)c2=O)c2cccnc21]

Acetic anhydride (0.023 mL, 0.247 mmol) was added to chloro-5-{[2-oxo-1-(1H-pyrazolo[3,4-b]pyridin-3-ylmethyl)-4-(trifluoromethyl)-1,2-dihydropyridin-3-yl]oxy}benzonitrile (100 mg, 0.224 mmol) in NMP (2 mL) at room temperature, followed 15 minutes later by the addition of N,N-dimethylpyridin-4-amine (27.4 mg, 0.224 mmol). After 30 minutes, the reaction mixture was filtered and the solid was washed with acetonitrile to afford the title compound as a white solid. ¹H NMR (DMSO-d6) $ 8.74 (dd, J=4.6 Hz, J=1.5 Hz, 1H), 8.22 (dd, J=8.0 Hz, J=1.6 Hz, 1H), 7.66 (d, J=7.3 Hz, 1H), 7.36 (m, 1H), 7.33 (dd, J=7.8 Hz, J=4.6 Hz, 1H), 7.12 (m, 1H), 6.90 (m, 1H), 6.45 (d, J=7.3 Hz, 1H), 5.51 (s, 2H), 2.92 (s, 3H). HRMS (M+1)=488.0741.

### Example 1

Potassium (3-{[3-(3-chloro-5-cyanophenoxy)-2-oxo-4-(trifluoromethyl)pyridin-1(2H)-yl]methyl}-1H-pyrazolo[3,4-b]pyridin-1-yl)methyl hydrogen phosphate monohydrate

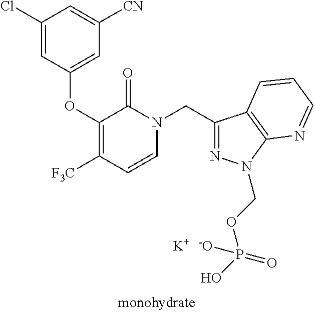

### Step 1: Di-tert-butyl chloromethyl phosphate

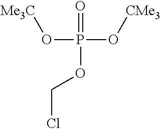

### Cl

Tetrabutylammonium hydrogen sulfate (1.216 g, 3.58 mmol) and sodium carbonate (12.03 g, 143 mmol) were added to barium bis(di-tert-butyl phosphate) (19.9 g, 35.8 mmol) dissolved in water (300 mL) at 0° C. CH₂Cl₂ (200 mL) was then added under N2. The mixture was cooled to 0° C. and, while stirring vigorously (i.e., stirring at a rate that provided a thorough mixing of the biphasic reaction mixture), chloromethyl sulfurochloridate (8.86 g, 53.7 mmol) was added drop-wise as a solution in CH₂Cl₂ (100 mL) over the course of 30 minutes under N2. The resulting mixture was then allowed to warm to room temperature and was stirred overnight under N2. After 16 hours, the reaction mixture was separated and the aqueous portion was washed with CH₂Cl₂ (250 mL). The combined organic fractions were washed with water (250 mL), dried (Na₂SO₄), after which solvent was removed by evaporation in vacuo. The resulting residue was purified by column chromatography on silica gel, eluting with 0-100% ethyl acetate/hexanes (visualized with a KMnO₄ stain after eluting with 50% ethyl acetate/hexanes (R_{f}=0.75) on TLC) to afford the title compound as a clear oil. ¹H NMR (CDCl₃) δ 5.65 (d, J=14.9 Hz, 2H), 1.50 (s, 18H). LRMS (M+1)=259.16.

### Step 2: Di-tert-butyl (3-{[3-(3-chloro-5-cyanophenoxy)-2-oxo-4-(trifluoromethyl)pyridin-1(2H)-yl]methyl}-1H-pyrazolo[3,4-b]pyridin-1-yl)methyl phosphate

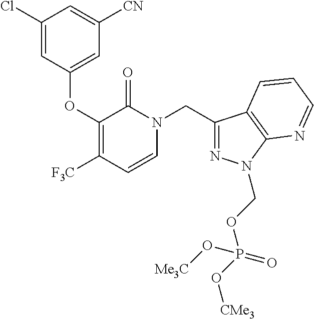

DMA (65 mL) was added to 3-chloro-5-{[2-oxo-1-(1H-pyrazolo[3,4-b]pyridin-3-ylmethyl)-4-(trifluoromethyl)-1,2-dihydropyridin-3-yl]oxy}benzonitrile (5.035 g, 11.29 mmol) and the resulting mixture was stirred under N2 until a solution was achieved. Cs₂CO₃ (7.36 g, 22.59 mmol) was then added and the mixture allowed to stir. After two minutes, di-tert-butyl chloromethyl phosphate (3.07 g, 11.86 mmol) was added as a solution in DMA (15 mL) under N2 at room temperature. After 23 hours, the reaction mixture was diluted with ethyl acetate (500 mL) and filtered. The filtrate was washed with dilute brine (4×500 mL). The extract was dried (Na₂SO₄) and filtered, and then solvent was removed by evaporation in vacuo. The resulting oil was purified by flash chromatography on basic alumina eluting with 0-50% ethyl acetate/hexanes to yield the title compound. ¹H NMR (CDCl₃) δ 8.60 (dd, J=4.6 Hz, J=1.6 Hz, 1H), 8.17 (dd, J=6.6 Hz, J=1.5 Hz, 1H), 7.68 (d, J=7.3, 1H), 7.40-7.36 (m, 1H), 7.23 (dd, J=4.5 Hz, J=3.6 Hz, 1H), 7.15-7.10 (m, 1H), 7.05-6.95 (m, 1H), 6.42 (d, J=7.3 Hz, 1H), 6.28 (d, J=8.6 Hz, 2H), 5.52 (s, 2H). LRMS (M+1)=668.3.

### Step 3: (3-{[3-(3-Chloro-5-cyanophenoxy)-2-oxo-4-(trifluoromethyl)pyridin-1(2H)-yl]methyl}-1H-pyrazolo[3,4-b]pyridin-1-yl)methyl dihydrogen phosphate

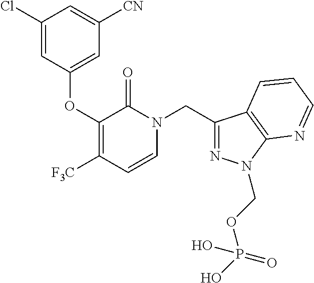 [SMILES: N#Cc1cc(Cl)cc(Oc2c(C(F)(F)F)ccn(Cc3nn(COP(=O)(O)O)c4ncccc34)c2=O)c1]

TFA (5 mL) was added to di-tert-butyl (3-{[3-(3-chloro-5-cyanophenoxy)-2-oxo-4-(trifluoromethyl)pyridin-1(2H)-yl]methyl}-1H-pyrazolo[3,4-b]pyridin-1-yl)methyl phosphate (3.25 g, 4.87 mmol) in dichloromethane (30 mL) to afford a solution. After 10 minutes, the solution was diluted with dichloromethane (200 mL) and then solvent was removed by evaporation in vacuo with no heating. The resulting concentrate was diluted with toluene (25 mL) and placed in a 30° C. water bath, and then solvent was removed by evaporation in vacuo while maintaining the water bath at 30° C. The resulting solid was suspended in acetonitrile and filtered to yield the title compound as a white solid. ¹H NMR (DMSO-d6) δ 8.62 (d, J=4.6 Hz, 1H), 8.21 (d, J=8.0 Hz, 1H), 8.09 (d, J=7.3 Hz, 1H), 7.75 (m, 1H), 7.58 (m, 1H), 7.52 (m, 1H), 7.32 (dd, J=8.0 Hz, J=4.6 Hz, 1H), 6.67 (d, J=7.3 Hz, 1H), 6.12 (d, J=6.6 Hz, 2H), 5.57 (s, 2H). LRMS (M+1)=556.0.

### Step 4: Potassium (3-{[3-(3-chloro-5-cyanophenoxy)-2-oxo-4-(trifluoromethyl)pyridin-1(2H)-yl]methyl}-1H-pyrazolo[3,4-b]pyridin-1-yl)methyl hydrogen phosphate monohydrate

To (3-{[3-(3-chloro-5-cyanophenoxy)-2-oxo-4-(trifluoromethyl)pyridin-1(2H)-yl]methyl}-H-pyrazolo[3,4-b]pyridin-1-yl)methyl dihydrogen phosphate (290 mg, 0.522 mmol)dissolved in methanol (20 mL) and dichloromethane (10 mL) was added a solution of potassium acetate (205 mg, 2.087 mmol) in methanol (5 mL) at room temperature. The reaction was allowed to stir at room temperature for 16 hours during which time a precipitate formed. This mixture was filtered and the recovered solid was washed with methanol and placed in a vacuum oven at 30 C under a stream of N2 to remove solvent, which afforded the title compound as a crystalline solid. ¹H NMR (DMSO-d6) δ 8.58 (dd, J=4.5, J=1.6 Hz, 1H), 8.15 (dd, J=8.0 Hz, J=1.5 Hz, 1H), 8.10 (d, J=7.1 Hz, 1H), 7.78 (m, 1H), 7.61 (m, 1H), 7.58 (m, 1H), 7.22 (dd, J=8.0 Hz, J=4.6 Hz, 1H), 6.62 (d, J=7.3 Hz, 1H), 5.98 (d, J=4.4 Hz, 2H), 5.57 (s, 2H). HRMS (M+1)=556.0399.

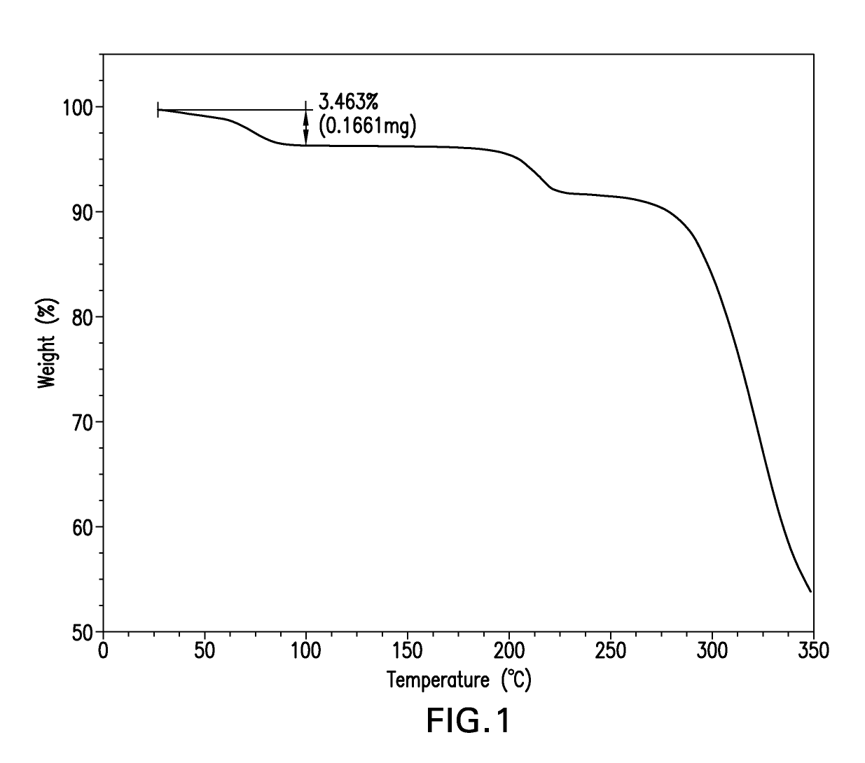

A TGA of the crystalline salt (sample size of approximately 5 mg) was performed using a TA Instruments Q5000 analyzer under nitrogen at a heating rate of 10° C./minute from 25° C. to 250° C. The TGA curve (see FIG. 1) showed a loss of 3.46 wt. % up to 100° C. which is consistent with the solid being a monohydrate. In a separate TGA run, the salt was heated from room temperature to 120° C. and then allowed to cool back to room temperature at ambient conditions and re-heated to 120° C. The salt was observed to rehydrate and dehydrate quantitatively during the cycle without a change in the crystal indicating the crystalline form is stable.

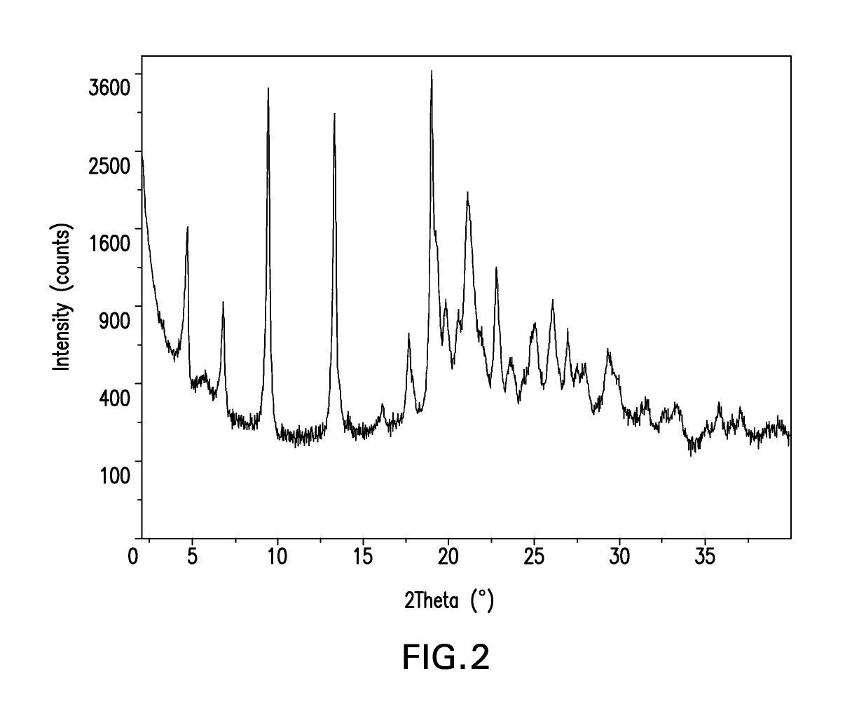

An XRPD pattern of the crystalline product was generated on a Philips Pananalytical X'Pert Pro X-ray powder diffractometer with a PW3373/10 Cu LFF DK184158 console using a continuous scan from 2.5 to 40 degrees 20. Copper K-Alpha 1 (K_{α1}) and K-Alpha 2 (K_{α2}) radiation was used as the source. Approximately 10 mg of sample was placed on a silicon disk, flattened and analyzed. The experiment was conducted with the sample at room temperature and open to the atmosphere. The XRPD pattern is shown in FIG. 2. 2Θ values, the corresponding d-spacings, and the relative peak intensities of the most representative diffraction peaks in the XRPD pattern include the following:

TABLE B
XRPD Data

| Peak | d-spacing | 2 | I/Imax |
|---|---|---|---|
| No. | (Å) | Theta | (%) |
| 1 | 18.9 | 4.67 | 33.0 |
| 2 | 13.0 | 6.78 | 16.6 |
| 3 | 9.35 | 9.46 | 92.0 |
| 4 | 6.63 | 13.3 | 82.0 |
| 5 | 5.02 | 17.7 | 14.1 |
| 6 | 4.67 | 19.0 | 100 |
| 7 | 4.59 | 19.3 | 35.9 |
| 8 | 4.48 | 19.8 | 21.8 |
| 9 | 4.21 | 21.1 | 51.1 |
| 10 | 3.90 | 22.8 | 30.9 |
| 11 | 3.76 | 23.6 | 10.1 |
| 12 | 3.56 | 25.0 | 17.4 |
| 13 | 3.42 | 26.0 | 22.1 |
| 14 | 3.31 | 26.9 | 15.3 |
| 15 | 3.05 | 29.3 | 11.8 |

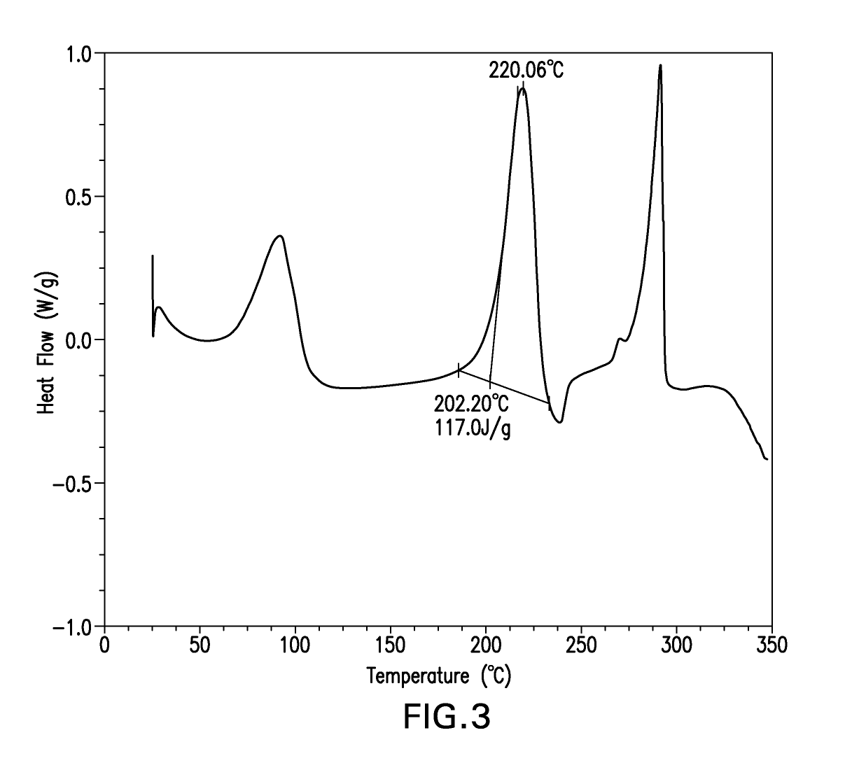

The crystalline product (ca. 5 mg) was analyzed with a TA Instruments DSC Q2000 differential scanning calorimeter (DSC) at a heating rate of 10° C./minute from 25° C. to 350° C. in an open aluminum pan in a nitrogen atmosphere. The DSC curve (see FIG. 3) exhibited an endotherm with an onset temperature of 202.2° C. and a peak temperature of 220.06° C. The enthalpy change was 117.0 J/g. The broad endotherm between 50° C. and 100° C. is attributed to loss of water/solvate.

### Example 2

### (3-{[3-(3-Chloro-5-cyanophenoxy)-2-oxo-4-(trifluoromethyl)pyridin-1(2H)-yl]methyl}-1H-pyrazolo[3,4-b]pyridin-1-yl)methyl dihydrogen phosphate ammoniate

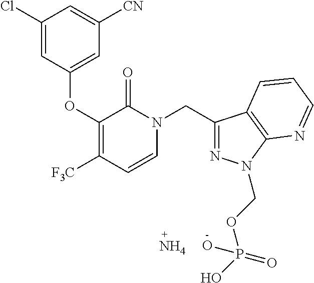

NH₄OH solution (−29%, 14 mL) was added to a suspension of (3-{[3-(3-chloro-5-cyanophenoxy)-2-oxo-4-(trifluoromethyl)pyridin-1(2H)-yl]methyl}-1H-pyrazolo[3,4-b]pyridin-1-yl)methyl dihydrogen phosphate (2.68 g, 4.82 mmol) in acetonitrile (135 mL). Water (65 mL) was then added to form a solution which was then filtered. Solvent was removed from the filtrate by evaporation in vacuo with the aid of acetonitrile as an azeotrope, and the resulting wet solid was suspended in acetonitrile (100 mL) and filtered to afford the title compound as a white solid. ¹H NMR (DMSO-d6) δ 8.55 (m, 1H), 8.15 (d, J=6.9 Hz, 1H), 8.08 (d, J=7.1 Hz, 1H), 7.72 (m, 1H), 7.58 (m, 1H), 7.52 (m, 1H), 7.22 (dd, J=8.0 Hz, J=4.4 Hz, 1H), 6.75 (br, 4H), 6.62 (d, J=7.3 Hz, 1H), 5.92 (d, J=4.1 Hz, 2H), 5.53 (s, 2H). LRMS (M+1)=556.1.

### Example 3

### Methyl 3-{[3-(3-chloro-5-cyanophenoxy)-2-oxo-4-(trifluoromethyl)pyridin-1(2H)-yl]methyl}-1H-pyrazolo[3,4-b]pyridine-1-carboxylate

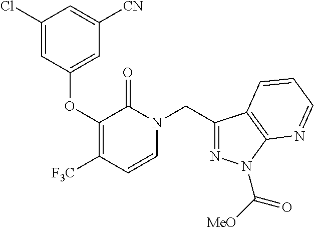 [SMILES: COC(=O)n1nc(Cn2ccc(C(F)(F)F)c(Oc3cc(Cl)cc(C#N)c3)c2=O)c2cccnc21]

3-chloro-5-{[2-oxo-1-(1H-pyrazolo[3,4-b]pyridin-3-ylmethyl)-4-(trifluoromethyl)-1,2-dihydropyridin-3-yl]oxy}benzonitrile (1.711 g, 3.84 mmol) was stirred at room temperature in DMA (20 mL) under N2 until a solution was achieved. Cs₂CO₃ (2.501 g, 7.68 mmol) was then added to the solution, and after 15 minutes methyl chloroformate (0.590 mL, 7.68 mmol) was then added, both additions conducted at room temperature. After 30 minutes, the reaction mixture was diluted with ethyl acetate (400 mL) and water was added (200 mL). The biphasic suspension was filtered and the recovered solid was washed with ethyl acetate (100 mL) and set aside. The biphasic filtrate was separated and the organic extract was washed with water (4×250 mL). The extract was dried (MgSO₄), filtered and the solvent was evaporated under reduced pressure. The resulting solid was adsorbed onto silica gel and purified by column chromatography eluting with 0-50% ethyl acetate/CH₂Cl₂ to afford a solid. This in combination with the previously filtered solid from the reaction mixture were dissolved in acetonitrile (5 mg/mL) with heating, after which the hot solution was filtered, and the filtrate allowed to crystallize. After 16 hours, the solids were collected by filtration to afford the title compound. ¹H NMR (DMSO-d6) δ 8.73 (dd, J=4.6 Hz, J=1.6 Hz, 1H), 8.32 (dd, J=8.0 Hz, J=1.7 Hz, 1H), 8.12 (d, J=7.1 Hz, 1H), 7.73 (m, 1H), 7.54 (m, 1H), 7.47 (m, 2H), 6.71 (d, J=7.3 Hz, 1H), 5.60 (s, 2H), 4.02 (s, 3H). HRMS (M+1)=504.0675.

### Example 4

### Ethyl 3-{[3-(3-chloro-5-cyanophenoxy)-2-oxo-4-(trifluoromethyl)pyridin-1(2H)-yl]methyl}-1H-pyrazolo[3,4-b]pyridine-1-carboxylate

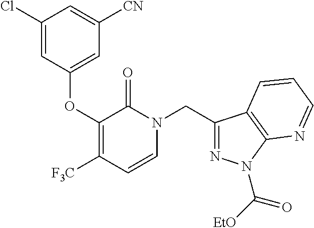 [SMILES: CCOC(=O)n1nc(Cn2ccc(C(F)(F)F)c(Oc3cc(Cl)cc(C#N)c3)c2=O)c2cccnc21]

3-chloro-5-{[2-oxo-1-(1H-pyrazolo[3,4-b]pyridin-3-ylmethyl)-4-(trifluoromethyl)-1,2-dihydropyridin-3-yl]oxy}benzonitrile (1.762 g, 3.95 mmol) was stirred in DMA (20 mL) at room temperature under N2 to afford a solution, to which was added Cs₂CO₃ (2.58 g, 7.91 mmol) and, after 15 minutes, ethyl chloroformate (0.753 mL, 7.91 mmol) to provide a reaction mixture. After 30 minutes, the reaction mixture was diluted with ethyl acetate (400 mL) and water was added (200 mL). The biphasic suspension was filtered and the solid washed with ethyl acetate (100 mL). The biphasic filtrate was separated and the organic extract was washed with water (4×250 mL). The extract was dried (MgSO₄), filtered and the solvent was evaporated under reduced pressure. The resulting solid was adsorbed onto silica gel and purified by column chromatography on silica eluting with 0-50% ethyl acetate/CH₂Cl₂ to yield a solid. This was combined with the previously filtered solid and dissolved in acetonitrile (2 mg/mL) with heating, hot filtered and allowed to crystallize. After 16 hours, the solid was collected by filtration to afford the title compound. ¹H NMR (DMSO-d6) δ 8.73 (dd, J=4.6 Hz, J=1.6 Hz, 1H), 8.32 (dd, J=8.0 Hz, J=1.7 Hz, 1H), 8.12 (d, J=7.1 Hz, 1H), 7.73 (m, 1H), 7.54 (m, 1H), 7.47 (m, 2H), 6.71 (d, J=7.3 Hz, 1H), 5.60 (s, 2H), 4.50 (q, J=7.1 Hz, 2H), 1.37 (t, J=7.1 Hz, 3H). HRMS (M+1)=518.0829.

### Example 5

### 2-{[(3-{[3-(3-Chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}-N-methylethanaminium chloride

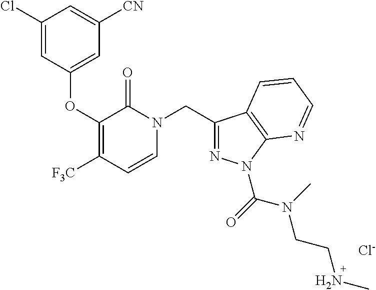 [SMILES: CNCCN(C)C(=O)n1nc(Cn2ccc(C(F)(F)F)c(Oc3cc(Cl)cc(C#N)c3)c2=O)c2cccnc21 ; [Cl-]]

### Step 1: tert-Butyl (2-{[(3-{[3-(3-chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethyl)methylcarbamate

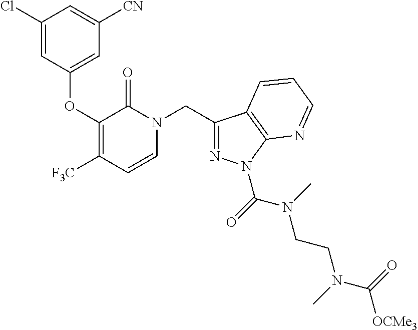

3-chloro-5-{[2-oxo-1-(1H-pyrazolo[3,4-b]pyridin-3-ylmethyl)-4-(trifluoromethyl)-1,2-dihydropyridin-3-yl]oxy}benzonitrile (1.20 g, 2.69 mmol) was allowed to stir in DMA (25 mL) under N2 at room temperature until a solution was achieved. The solution was cooled to 0° C. and then treated with N-ethyl-N-(propan-2-yl)propan-2-amine (0.940 mL, 5.38 mmol) followed by 4-nitrophenyl carbonochloridate (651 mg, 3.23 mmol). The mixture was allowed to warm to room temperature, and was then stirred for 30 minutes. The reaction mixture was then cooled to 0° C. and tert-butyl methyl[2-(methylamino)ethyl]carbamate (608 mg, 3.23 mmol) in DMA (4 mL) was added, after which the mixture was allowed to warm to room temperature. After 1 hour, the reaction mixture was diluted with ethyl acetate (200 mL), washed with water (4×50 mL), dried (MgSO₄), and filtered, and then the solvent was evaporated in vacuo to afford a solid. The solid was purified by column chromatography eluting with 0-100% ethyl acetate/CH₂Cl₂ to afford the title compound. ¹H NMR (CDCl₃) δ 8.67 (d, J=3.8 Hz, 1H), 8.21 (m, 1H), 7.65 (m, 1H), 7.39 (m, 1H), 7.28 (m, 1H), 7.15 (m, 1H), 7.02 (m, 1H), 6.45 (m, 1H), 5.52 (s, 2H), 3.8-3.5 (m, 5H), 3.3-2.9 (m, 5H), 1.45 (s, 9H). LRMS (M+1)=660.0.

### Step 2: 2-{[(3-{[3-(3-Chloro-5-cyanophenoxy)-2-oxo-4-(trifluoromethyl)pyridin-1(2)-yl]methyl}-1H-pyrazolo[3,4-b]pyridin-1-yl)carbonyl](methyl)amino}-N-methylethanaminium chloride

tert-Butyl (2-{[(3-{[3-(3-chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethyl)methylcarbamate (460 mg, 0.697 mmol) was dissolved in TFA (2 mL). After 10 minutes solvent was removed from the reaction mixture by evaporation in vacuo and the resulting residue was diluted with acetonitrile, filtered and purified by preparative HPLC, eluting with 5-95% MeCN/H₂O+0.1% TFA. This was lyophilized to afford a solid, which was dissolved in absolute ethanol (30 mL). HC (4M in dioxane; 0.668 mL, 2.67 mmol) was then added to the solution, and then solvent was removed from the solution by evaporation in vacuo to afford a solid. ¹H NMR (DMSO-d6) δ 8.82 (m, 2H), 8.67 (dd, J=4.7 Hz, J=1.5 Hz, 1H), 8.36 (m, 1H), 8.20 (m, 1H), 7.75 (m, 1H), 7.58 (m, 1H), 7.53 (m, 1H), 7.42 (dd, J=8.0 Hz, J=4.6 Hz, 1H), 6.71 (d, J=7.3 Hz, 1H), 5.62 (s, 2H), 3.70 (m, 2H), 3.20 (m, 2H), 3.01 (m, 3H), 2.58 (m, 3H). HRMS (M+1)=506.1410.

### Example 6

### 2-{[(3-{[3-(3-Chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethanaminium chloride

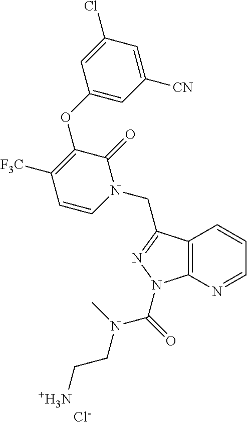 [SMILES: CN(CC[NH3+])C(=O)n1nc(Cn2ccc(C(F)(F)F)c(Oc3cc(Cl)cc(C#N)c3)c2=O)c2cccnc21 ; [Cl-]]

To a solution of 2-{[(3-{[3-(3-chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethanaminium trifluoroacetate (see Preparative Example 3) (3.86 g, 5.98 mmol) in CH₂Cl₂ (50 mL) was added a 4M solution of hydrochloric acid (3 mL, 11.95 mmol). Solvent was removed from the resulting solution by evaporation in vacuo. The resulting residue was suspended in ethanol (150 mL) and treated with additional 4M hydrochloric acid in dioxane (4.5 mL, 18 mmol) and the resulting suspension was heated to 70° C. for 20 minutes. The suspension was then allowed to cool to room temperature and filtered. The solid was washed with ethanol (30 mL) at room temperature, air dried for 1 hour, and then dried under high vacuum for 4 days. ¹H NMR (DMSO-d6) δ=8.67 (d, 1H, J=1.5 and 4.6 Hz), 8.36 (d, 1H, J=6.3 Hz), 8.18 (br m, 1H), 7.95 (br s, 3H), 7.75 (d, 1H, J=1.5 Hz), 7.57 (m, 1H), 7.52 (m, 1H), 7.42 (dd, 1H, J=4.6 and 8 Hz), 6.71 (d, 1H, J=7.3 Hz), 5.61 (s, 2H), 3.64 (br s, 2H), 3.10 (br s, 2H) and 3.03 (br s, 3H) ppm. F-NMR (DMSO-d6) 8=−62.6 ppm. MS (M+1): 546.1.

### Example 7

### 2-{[(3-{[3-(3-Chloro-5-cyanophenoxy)-2-oxo-4-(trifluoromethyl)pyridin-1(2-yl]methyl}-1H-pyrazolo[3,4-b]pyridin-1-yl)carbonyl](methyl)amino}ethanaminium methanesulfonate

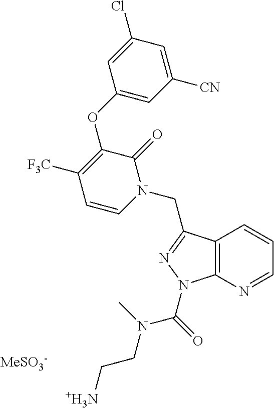 [SMILES: "MeSO3-" ; CN(CC[NH3+])C(=O)n1nc(Cn2ccc(C(F)(F)F)c(Oc3cc(Cl)cc(C#N)c3)c2=O)c2cccnc21]

To a suspension of 2-{[(3-{[3-(3-chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethanaminium trifluoroacetate (see Preparative Example 3) (203.1 mg, 0.308 mmol) in ethanol (5 mL) was added methanesulfonic acid (0.021 mL, 0.323 mmol) and the resulting suspension was slightly warmed until a solution was obtained, and then solvent was removed by evaporation in vacuo. This residue was dissolved in THF (3 mL) and then hexanes (1 mL) were added to form a cloudy mixture. This mixture was gently warmed until a solution was obtained, and then the solution was allowed to stand at room temperature for 3 days. After this time, the resulting white crystalline solid was collected by filtration and dried under high vacuum for 1.5 hours. This solid was suspended in ethyl acetate (20 mL) and mixed on a rotary evaporator for 10 minutes at 60° C. without a vacuum. This suspension was concentrated in vacuo (5 mL) and the resulting crystalline solid was collected by filtration and washed with ethyl acetate (2 mL) and dried under high vacuum for 2.5 hours to give the title compound. ¹H NMR (DMSO-d6) δ=8.67 (dd, 1H, J=1.5 and 4.6 Hz), 8.36 (br d, 1H), 8.15 (br d, 1H, J=6.8 Hz), 7.8 (br s, 3H), 7.75 (d, 1H, J=1.5 Hz), 7.57 (m, 1H), 7.52 (m, 1H), 7.42 (dd, 1H, J=4.6 and 8 Hz), 6.70 (d, 1H, J=7.3 Hz), 5.61 (s, 2H), 3.63 (br s, 2H), 3.10 (br s, 2H), 3.02 (br s, 3H) and 2.29 (s, 3H) ppm. LRMS (M+1): 546.1.

### Example 8

### 2-{[(3-{[3-(3-Chloro-5-cyanophenoxy)-2-oxo-4-(trifluoromethyl)pyridin-1(2H)-yl]methyl}-1H-pyrazolo[3,4-b]pyridin-1-yl)carbonyl](methyl)amino}ethanaminium 4-methylbenzenesulfonate

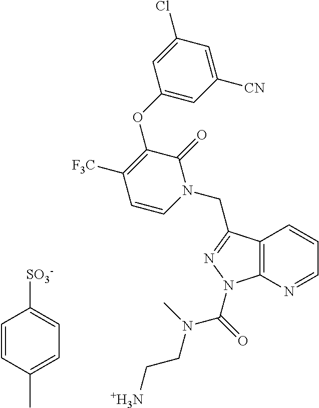

To a suspension of 2-{[(3-{[3-(3-chloro-5-cyanophenoxy)-2-oxo-4-(trifluoromethyl)pyridin-(2H)-yl]methyl}-1H-pyrazolo[3,4-b]pyridin-1-yl)carbonyl](methyl)amino}ethanaminium trifluoroacetate (see Preparative Example 3) (202.1 mg, 0.323 mmol) in ethanol (5 mL) was added p-toluenesulfonic acid hydrate (64.4 mg, 0.339 mmol) and the resulting suspension was gently warmed until a solution was obtained, after which solvent was removed by evaporation in vacuo. The resulting residue was dissolved in THF (3 mL) and then hexanes (2 mL) were added to afford a cloudy mixture, which was then gently warmed but did not redissolve. Additional THF (3 mL) was added after which the mixture was warmed to and held at 60° C. until the residue dissolved. The solution was then allowed to stand at room temperature for 3 days, after which the resulting white crystalline solid was collected by filtration and dried under high vacuum for 1.5 hours. This solid was suspended in ethyl acetate (20 mL) and mixed on a rotary evaporator for 10 minutes under no vacuum with the water bath maintained at 60° C. The suspension was then concentrated in vacuo (5 mL) and the resulting crystalline solid was collected by filtration, washed with ethyl acetate (2 mL), and dried under high vacuum for 18 hours to give the title compound as a white crystalline solid. ¹H NMR (DMSO-d6) δ=8.67 (m, 1H), 8.36 (m, 1H), 8.15 (br d, 1H), 7.8 (br s, 3H), 7.75 (m, 1H), 7.57 (m, 1H), 7.52 (m, 1H), 7.47 (d, 2H, J=8 Hz), 7.42 (dd, 1H, J=4.6 and 8 Hz), 7.10 (d, 2H, J=8 Hz), 6.70 (d, 1H, J=7.3 Hz), 5.61 (s, 2H), 3.63 (br s, 2H), 3.10 (br s, 2H), 3.02 (br s, 3H) and 2.29 (s, 3H) ppm. LRMS (M+1): 546.1.

### Example 9

### N-(2-Aminoethyl)-3-{[3-(3-chloro-5-cyanophenoxy)-2-oxo-4-(trifluoromethyl)pyridin-1(2H)-yl]methyl}-N-methyl-1H-pyrazolo[3,4-b]pyridine-1-carboxamide benzenesulfonate

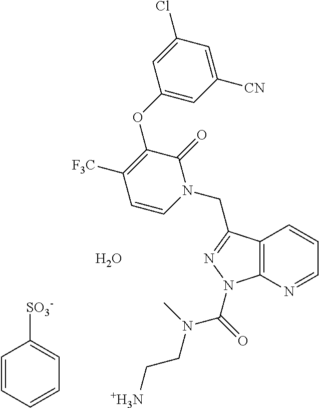

To a suspension of 2-{[(3-{[3-(3-chloro-5-cyanophenoxy)-2-oxo-4-(trifluoromethyl)pyridin-1(2H)-yl]methyl}-1-1H-pyrazolo[3,4-b]pyridin-1-yl)carbonyl](methyl)amino}ethanaminium trifluoroacetate (see Preparative Example 3) (12.81 g, 19.41 mmol) in acetonitrile (400 mL) was added benzenesulfonic acid (3.07 g, 19.41 mmol) and the resulting solution was evaporated in vacuo. The residue was redissolved in ethanol (300 mL) and the solvent removed by evaporation in vacuo. The redissolution in and evaporation of ethanol was repeated. The resulting solid was then partially dissolved in ethanol (200 mL), warmed over a water bath at 55° C. for 10 minutes and then allowed to stand at room temperature for 1 hour. The resulting white, crystalline solid was filtered and washed with ethanol (100 mL), air dried for 30 minutes, and then dried under high vacuum for 18 hours. The dried solid was then added to preheated acetonitrile (2650 mL) at 55° C. and stirred for 5 minutes while maintaining the temperature at 55° C. The resulting solution was then hot filtered and concentrated in vacuo to a weight of 500 g. This mixture was then filtered hot and concentrated in vacuo to a weight of 315 g. The concentrate was seeded with authentic crystalline salt and allowed to stand at room temperature for 3.5 hours. (Note: The concentrate could have been crystallized without the use of seed.) The resulting white solid was then collected by filtration, washed with acetonitrile (100 mL), air dried for 10 minutes, and then dried under high vacuum for 19 hours to give the title compound. ¹H NMR (DMSO-d6) δ=8.67 (dd, 1H, J=1.5 and 4.6 Hz), 8.36 (br d, 1H), 8.15 (br d, 1H, J=6.8 Hz), 7.8 (br s, 3H), 7.75 (d, 1H, J=1.5 Hz), 7.62-7.57 (m, 3H), 7.52 (dd, 1H, J=2 Hz), 7.42 (dd, 1H, J=4.6 and 8 Hz), 7.33-7.27 (m, 3H), 6.70 (d, 1H, J=7.3 Hz), 5.61 (s, 2H), 3.63 (br s, 2H), 3.10 (br s, 2H and 3.02 (br s, 3H) ppm. LRMS (M+1): 546.1.

### Example 9A

### N-(2-Aminoethyl)-3-{[3-(3-chloro-5-cyanophenoxy)-2-oxo-4-(trifluoromethyl)pyridin-[(2H)-yl]methyl}-N-methyl-1H-pyrazolo[3,4-b]pyridine-1-carboxamide benzenesulfonate hydrate

A mixture of N-(2-aminoethyl)-3-{[3-(3-chloro-5-cyanophenoxy)-2-oxo-4-(trifluoromethyl)pyridin-1(2H)-yl]methyl}-N-methyl-1H-pyrazolo[3,4-b]pyridine-1-carboxamide benzenesulfonate (0.1 g, 0.14 mmol) and water (5 mL) was stirred in a beaker at 700+ rpm. The mixture was allowed to equilibrate for 24 hours. After 24 hours, the mixture was filtered under vacuum onto filter paper using a Büchner funnel. The isolated solid was allowed to dry overnight at room temperature to afford crystals of the title solid. ¹H NMR (DMSO-d6) δ=8.67 (d, J=4.58 Hz, 1H), 8.35 (m, 1H), 8.15 (m, 1H), 7.80 (m, 3H), 7.75 (s, 1H), 7.55 (m, 3H), 7.47 (s, 1H), 7.40 (dd, J=8.1, Hz J=4.8 Hz, 1H), 7.30 (m, 3H), 6.68 (d, J=7.33 Hz, 1H), 5.61 (s, 2H), 3.82 (m, 2H), 3.15 (m, 2H), 3.00 (m, 3H). MS (M+1): 546.1

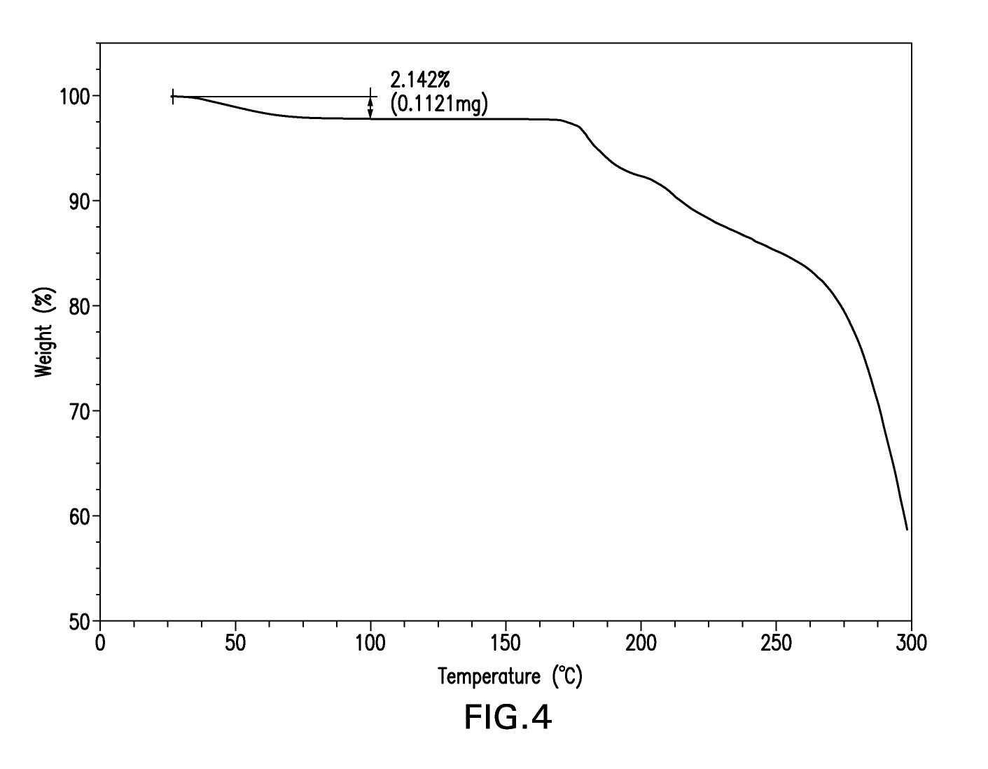

TGA analysis of the title solid (approximately 5 mg) was performed using a TA Instruments Q5000 analyzer under nitrogen at a heating rate of 10° C./minute from 25° C. to 300° C. The TGA curve (see FIG. 4) showed a loss of 2.14 wt. % up to 100° C. which is consistent with the solid being a monohydrate.

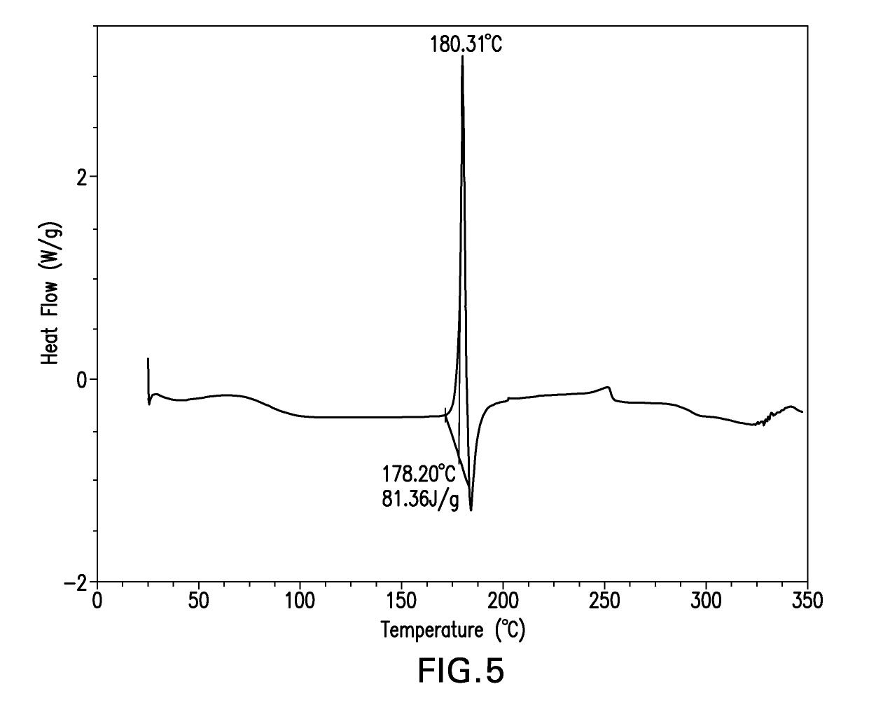

DSC analysis of the title solid was performed using a TA Instruments Q2000 analyzer from 25° C. to 300° C. at a heating rate of 10° C./min. The DSC curve (see FIG. 5) shows a melting endotherm at 180.3° C. with a heat of fusion of 81.4 J/g. Also, a broad endotherm between 50° C. to 100° C. was seen, which can be attributed to loss of water/solvate.

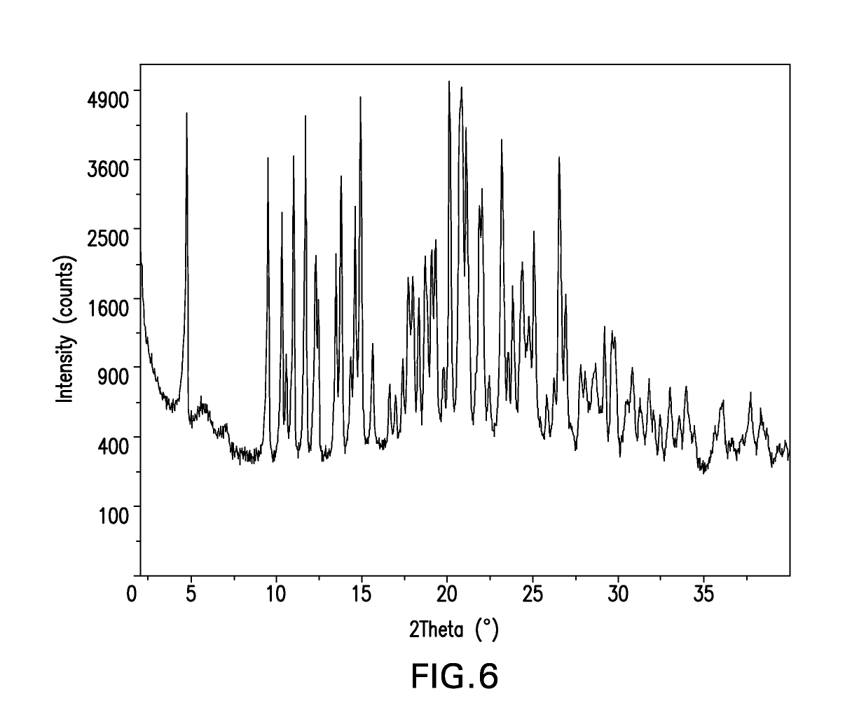

An XRPD pattern of the crystalline product was generated on a Philips Pananalytical X'Pert Pro X-ray powder diffractometer with a PW3373/10 Cu LFF DK184158 console using a continuous scan from 2.5 to 40 degrees 2Θ. Copper K-Alpha 1 (K_{α1}) and K-Alpha 2 (K_{α2}) radiation was used as the source. Approximately 10 mg of sample was placed on a silicon disk, flattened and analyzed. The experiment was conducted with the sample at room temperature and open to the atmosphere. The XRPD pattern is shown in FIG. 6. 2Θ values, the corresponding d-spacings, and the relative peak intensities of the most representative diffraction peaks in the XRPD pattern include the following:

TABLE C
XRPD Data

| Peak | d-spacing | 2 | I/Imax |
|---|---|---|---|
| No. | (Å) | Theta | (%) |
| 1 | 18.7 | 4.73 | 78.7 |
| 2 | 9.32 | 9.49 | 67.9 |
| 3 | 8.59 | 10.3 | 48.7 |
| 4 | 8.05 | 11.0 | 69.7 |
| 5 | 7.57 | 11.7 | 83.3 |
| 6 | 6.44 | 13.8 | 62.8 |
| 7 | 6.07 | 14.6 | 52.6 |
| 8 | 5.94 | 14.9 | 92.3 |
| 9 | 4.29 | 20.7 | 82.9 |
| 10 | 4.26 | 20.8 | 94.1 |
| 11 | 4.08 | 21.8 | 51.5 |
| 12 | 4.04 | 22.0 | 58.8 |
| 13 | 3.84 | 23.1 | 75.0 |
| 14 | 3.56 | 25.0 | 44.6 |
| 15 | 3.37 | 26.5 | 66.9 |

### Example 10

### Pharmacokinetic and Solubility Studies

Pharmacokinetic (PK) values for Compound A were determined in Wistar-Hannover rats (three male rats from Taconic Farms) orally dosed with the compound of Example 1 prepared as a solution in 10% Tween. The rats were housed in an AAALAC-accredited facility in accordance with USDA guidelines. Studies were conducted under a protocol approved by an internal animal care and use committee. Rat weights, measured and recorded prior to dosing, ranged from approximately 250 to 300 g. The dose of the compound of Example 1 was adjusted to deliver Compound A at approximately 10 mg per kg of body weight (i.e., 10 mpk)

Dosing:

After an overnight fast, the rats received the liquid formulations dosed orally at 5 mL/kg. The animals were allowed free access to water, and food was returned 4 hours after dosing. Blood was drawn from catheters placed in the femoral vein at pre-dose, and 0.25, 0.5, 1, 2, 4, 6, 8 and 24 hours after dosing. The plasma was separated by centrifugation (10 minutes at 2500 g) and stored overnight at −70° C. for LC/MS/MS the following day.

Sample Preparation and Analysis:

LC-MS-MS analysis was performed, wherein the plasma samples were extracted using automated (Hamilton Liquid Handler) protein precipitation, centrifugation and transfer of supernatants to analysis plates. Aliquots of the supernatants were injected on an Ascentis Express C18 (50×2.1 mm×2.7 u). The sample extracts were ionized using an ESI interface and samples were monitored by selected reaction monitoring (SRM) in the positive ionization mode. The dynamic range of the LC/MS/MS assay was 2-5,000 nM based on a 50 μL aliquot of rat plasma. In addition, chromatographic peaks for the compound of Example 1 and Compound A were resolved chromatographically with baseline to baseline separation.

PK Calculations:

Pharmacokinetic parameters were obtained using non-compartmental methods (Watson®). The area under the plasma concentration-time curve (AUC₀₋ₜ) was calculated from the first time point (0 minutes) up to the last time point with measurable drug concentration using the linear trapezoidal or linear/log-linear trapezoidal rule. The remaining area under the plasma concentration-time curve (AUC_{t-∞}) was estimated by dividing the observed concentration at the last time point by the elimination rate constant. This value was added to AUC₀₋ₜ to estimate the AUC_{0-∞}. The percentage AUC extrapolated was a function of (AUC_{0-∞}-AUC₀₋ₜ) 100/AUC_{0-∞}. The IV plasma clearance was calculated by dividing the dose by AUC_{0-∞}. The terminal half-life of elimination was determined by unweighted linear regression analysis of the log-transformed data. The time points for determination of half-life were selected by visual inspection of the data. The volume of distribution at steady state (V_{dss}) was obtained from the product of plasma clearance and mean residence time (determined by dividing the area under the first moment curve by the area under the curve). The maximum plasma concentration (Cₘₐₓ) and the time at which maximum concentration occurred (Tₘₐₓ) were obtained by inspection of the plasma concentration-time data. Absolute oral bioavailability was determined from dose-adjusted IV and P.O. exposure (AUC_{0-∞} or AUC₀₋ₗₐₛₜ if more than 20% of AUC_{0-∞} was extrapolated) ratios.

PK values were obtained in rats orally dosed with the following compounds prepared as a suspension in 0.5% methocel: Compound A, the compound of Examples C-1, and the compounds of Examples 1, 3-7 and 9. Table D provides the AUC values obtained in these studies.

TABLE D
Results of PK Studies

| | Rat AUC₀₋₂₄ hrs | |
|---|---|---|
| | (μM hr) @ | |
| Example | 0.022 mmol/kg PO¹ | Vehicle² |
| Compound A | 3.0 | 0.5% methocel |
| C-1 | 1.7 | 0.5% methocel |
| 1 | 25 | 0.5% methocel |
| 1 | 33 | 10% Tween 80 |
| 3 | 19 | 0.5% methocel |
| 4 | 7 | 0.5% methocel |
| 5 | 37 | 0.5% methocel + |
| | | 5 mM HCl |
| 6 | 35 | 0.5% methocel + |
| | | 5 mM HCl |
| 7 | 39 | 50 mM citric acid |
| 9 | 33 | 10% Tween 80 + |
| | | 50 mM citric acid |

1. PO = per os which is Latin for by mouth.
2. Acid was employed in certain tests to stabilize the compound.

### Solubility Studies

The water solubilities of Compound A and the title compounds of Examples 1 and 9 were determined at room temperature as follows: An excess amount of the compound was added to a microcentrifuge tube containing about 1 mL of deionized water. A stir bar was then placed in the tube and the tube placed on a multi-position stirrer plate. The sample was stirred continuously for a period of time (i.e., Compound A and Example 9 for 24 hours; Example 1 for 4 hours), after which the sample was spun at 15,000 rpm for 10 minutes. The solubility of each compound was determined via HPLC, wherein a portion of the supernatant was removed from the tube, diluted with ACN:water (50:50 by volume) as necessary to achieve a suitable concentration for analysis, and then analyzed by HPLC against a standard (=a solution of the K salt of Example 1 in ACN:water 50:50 by volume). One determination was made for each compound. The results are shown in Table E below.

HPLC: column=Agilent Zorbax Eclipse (4.6×50 mm, 1.8 micron); mobile phase=0.1% H₃PO₄:ACN; gradient=90% to 10% H₃PO₄; flow rate=1.5 mL/minute; column temperature=45° C.; injection volume=25 μL; UV sampling with detection wavelength=210 nm; run time=15 minutes.

TABLE E
Results of Solubility Studies

| | Water Solubility |
|---|---|
| Example | (mg/mL) |
| Compound A | 0.0002 |
| 1 | 12.0 |
| 9 | 1.1 |

While the foregoing specification teaches the principles of the present invention, with examples provided for the purpose of illustration, the practice of the invention encompasses all of the usual variations, adaptations and/or modifications that come within the scope of the following claims. All publications, patents and patent applications cited herein are incorporated by reference in their entireties into the disclosure.

1. A compound of Formula I: 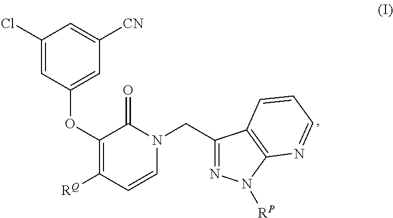
wherein:
R^{P} is:
(1) a base salt of 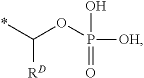
(2) an acid salt of C(O)N(R^{A})—C₁₋₆ alkylene-N(R^{A})R^{B};
(3) an acid salt of C(O)—C₁₋₆ alkylene-N(R^{A})R^{B}, or
(4) C(O)OR^{C};
R^{A} and R^{B} are each independently H or C₁₋₄ alkyl;
R^{C} is C₁₋₃ alkyl;
R^{D} is H or CH₃; and
R^{Q} is CF₃ or halogen.

2. (canceled)

3. A compound according to claim 1, wherein R^{Q} is CF₃.

4. (canceled)

5. A compound according to any one of claims 1 to 4, wherein R^{P} is:
(1) an alkali metal salt, an alkaline earth metal salt, an ammonium salt, or a tetra (C₁₋₄ alkyl) ammonium salt of 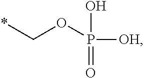 [SMILES: *COP(=O)(O)O]
(2) an alkali metal salt, an alkaline earth metal salt, an ammonium salt, or a tetra (C₁₋₄ alkyl) ammonium salt of 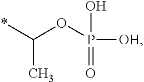 [SMILES: *C(C)OP(=O)(O)O]
(3) an acid salt of 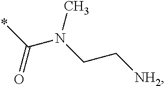 [SMILES: *C(=O)N(C)CCN]
(4) an acid salt of 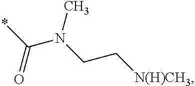
(5) C(O)CH₂N(CH₃)₂,
(6) C(O)C(CH₃)₂CH₂N(CH₃)₂,
(7) C(O)OCH₃, or
(8) C(O)OCH₂CH₃.

6. A compound according to claim 5, wherein R^{P} is an ammonium salt or an alkali metal salt of

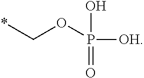 [SMILES: *COP(=O)(O)O]

7. (canceled)

8. A compound according to claim 6, wherein R^{P} is a sodium salt of

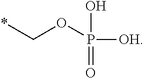 [SMILES: *COP(=O)(O)O]

9. A compound according to claim 6, wherein R^{P} is a potassium salt of

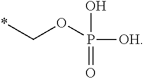 [SMILES: *COP(=O)(O)O]

10. A compound according to claim 9, wherein the potassium salt is a monopotassium salt or a dipotassium salt.

11. (canceled)

12. A compound according to claim 10, wherein R^{Q} is CF₃; and the potassium salt is a crystalline monopotassium monohydrate salt.

13. (canceled)

14. A compound according to claim 5, wherein R^{P} is selected from: C(O)CH₂N(CH₃)₂, C(O)C(CH₃)₂CH₂N(CH₃)₂, C(O)CH₂CH₂N(CH₃)₂, C(O)OCH₃ and C(O)OCH₂CH₃.

15-17. (canceled)

18. A compound according to claim 5, wherein R^{P} is an acid salt of

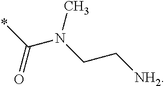 [SMILES: *C(=O)N(C)CCN]

19. A compound according to claim 18, wherein the acid salt is a hydrochloride salt, a besylate salt, a mesylate salt, a sulfonate salt, or a tosylate salt.

20-25. (canceled)

26. A compound according to claim 5, wherein R^{P} is an acid salt of

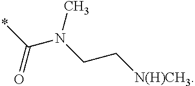

27. A compound according to claim 26, wherein the acid salt is a hydrochloride salt, a besylate salt, a mesylate salt, a sulfonate salt, or a tosylate salt.

28-32. (canceled)

33. A compound according to claim 1, which is a compound selected from the group consisting of:

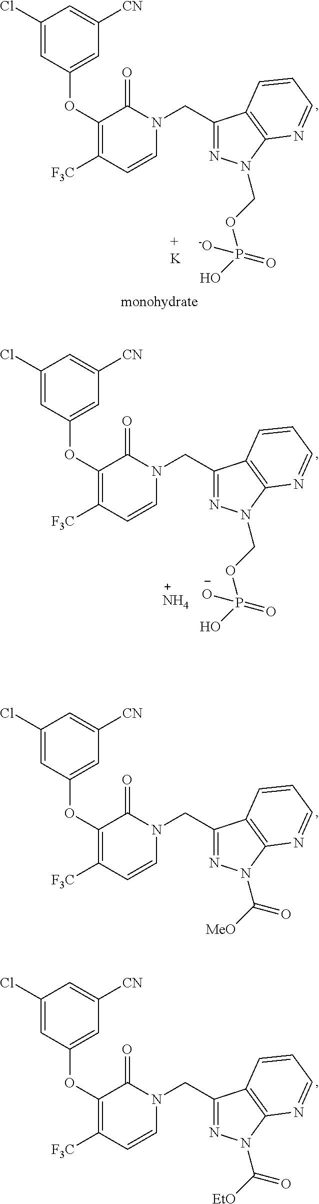

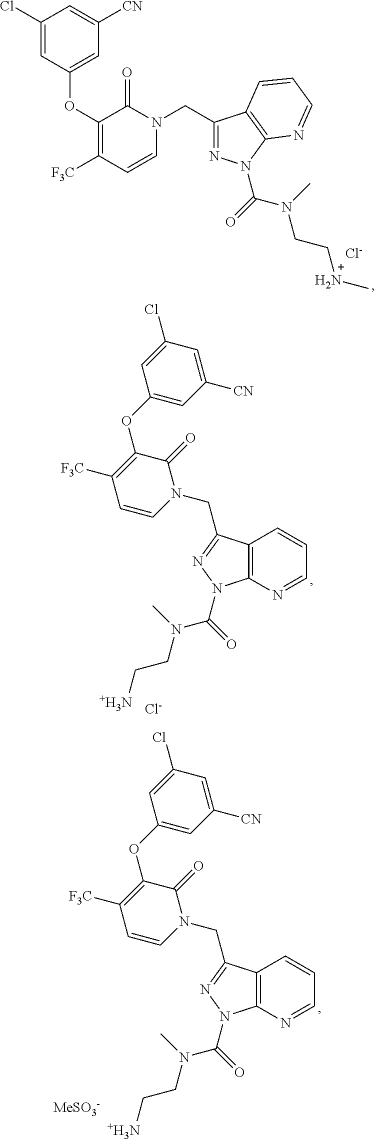 [SMILES: C[NH2+]CCN(C)C(=O)n1nc(Cn2ccc(C(F)(F)F)c(Oc3cc(Cl)cc(C#N)c3)c2=O)c2cccnc21 ; [Cl-] ; "MeSO3-" ; CN(CC[NH3+])C(=O)n1nc(Cn2ccc(C(F)(F)F)c(Oc3cc(Cl)cc(C#N)c3)c2=O)c2cccnc21 ; CN(CC[NH3+])C(=O)n1nc(Cn2ccc(C(F)(F)F)c(Oc3cc(Cl)cc(C#N)c3)c2=O)c2cccnc21 ; [Cl-]]

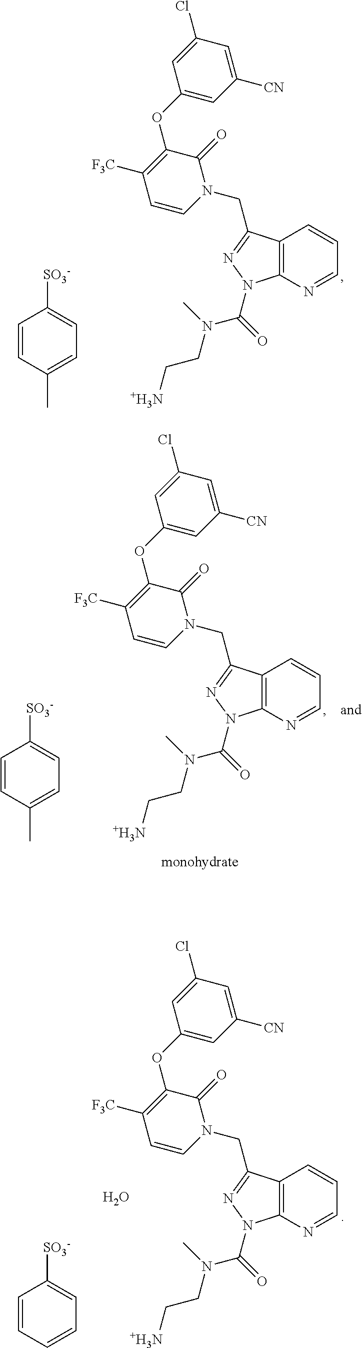

34. A pharmaceutical composition comprising a compound according to claim 1 and a pharmaceutically acceptable carrier.

35. A method for the inhibition of HIV reverse transcriptase, for the treatment or prophylaxis of HIV infection, or for the treatment, prophylaxis or delay in the onset of AIDS in a subject in need thereof, which comprises administering to the subject an effective amount of a compound according to any one of claim 1.

36. (canceled)
